(19) United States
(12) Patent Application Publication
(10) Pub. No.: US 2020/0277262 A1
(43) Pub. Date: Sep. 3, 2020

(54) N-(CYANO-SUBSTITUTED BENZYL OR PYRIDINYLMETHYL)-3-HYDROXYPICOLINAMIDE DERIVATIVES

(71) Applicants: Benjamin Jones, San Diego, CA (US); Andre Kiryanov, San Diego, CA (US); Jon Kuehler, San Diego, CA (US); Marion Lanier, San Diego, CA (US); Sean Murphy, San Diego, CA (US); Feng Zhou, San Diego, CA (US); Takeda Pharmaceutical Company Limited, Osaka (JP)

(72) Inventors: Benjamin Jones, Cardiff-by-the-Sea, CA (US); Andre Kiryanov, San Diego, CA (US); Jon Kuehler, San Diego, CA (US); Marion Lanier, San Diego, CA (US); Sean Murphy, San Diego, CA (US); Feng Zhou, San Diego, CA (US)

(21) Appl. No.: 16/650,858

(22) PCT Filed: Sep. 24, 2018
(86) PCT No.: PCT/US2018/052482

§ 371 Date: Mar. 25, 2020

Related U.S. Application Data
(60) Provisional application No. 62/562,969, filed on Sep. 25, 2017.

(51) Int. Cl. C07D 213/81

(52) U.S. Cl. CPC C07D 213/81; A61K 45/06

(57) ABSTRACT

Disclosed are compounds of Formula 1,

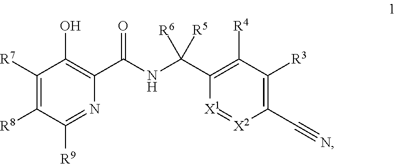

and pharmaceutically acceptable salts thereof, wherein R³, R⁴, R⁵, R⁶, R⁷, R⁸, R⁹, X¹ and X² are defined in the specification. This disclosure also relates to materials and methods for preparing compounds of Formula 1, to pharmaceutical compositions which contain them, and to their use for treating diseases, disorders, and conditions associated with PHD.

### FIELD OF THE INVENTION

This invention relates to N-(cyano-substituted benzyl or pyridinylmethyl)-3-hydroxypicolinamide derivatives which are inhibitors of hypoxia-inducible factor (HIF) prolyl hydroxylase, to pharmaceutical compositions which contain them, and to their use to treat diseases, disorders, and conditions associated with HIF prolyl hydroxylase.

### BACKGROUND OF THE INVENTION

Hypoxia-inducible factor (HIF) mediates gene expression in response to changes in cellular oxygen concentration. HIF is a heterodimer having an oxygen-regulated subunit (HIF-α) and a constitutively expressed subunit (HIF-β). HIF prolyl hydroxylase, which is also known as prolyl hydroxylase domain-containing protein (PHD), exists as three isoforms in humans (PHD1, PHD2, and PHD3). Together with HIF, the PHD enzyme regulates cellular metabolism in response to cellular oxygen level. In cells with adequate oxygen, HIF prolyl hydroxylase catalyzes the hydroxylation of conserved proline residues on HIF-α, resulting in rapid degradation of the transcription factor. Since oxygen is a cosubstrate for PHD enzymatic activity, HIF-α avoids degradation under hypoxic conditions, so it can translocate into the nucleus where it dimerizes with HIF-β. The resulting functional HIF complex activations transcription of various genes, including those encoding erythropoietin, vascular endothelial growth factor, and other proteins of biological interest. Because of the central role HIF prolyl hydrolase plays in cellular oxygen sensing, inhibitors of PHD may be useful in treating cardiovascular disorders, metabolic disorders, hematological disorders, pulmonary disorders, kidney disorders, liver disorders, would healing disorders, and cancer, among others.

### SUMMARY OF THE INVENTION

This invention provides N-(cyano-substituted benzyl or pyridinylmethyl)-3-hydroxypicolinamide derivatives and pharmaceutical compositions which contain them. The 3-hydroxypicolinamide derivatives are inhibitors of hypoxia-inducible factor (HIF) prolyl hydroxylase modulators (PHD) and may be used to treat diseases, disorders, and conditions associated with PHD.

One aspect of the invention provides a compound of Formula 1:

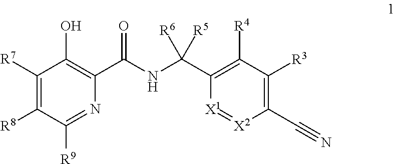

or a pharmaceutically acceptable salt thereof in which:
X¹ is selected from N and CR¹, and
X² is selected from N and CR², provided:
   (a) no more than one of X¹ and X² is N, and
   (b) when R⁴ and R⁵ are linked to form an ethane-1,2-diyl, a propane-1,3-diyl or a methane-1,1-diyloxy, then X¹ is CR¹ and X² is CR², and
   (c) when X¹ is CR¹ and X² is CR², then at least one of R, R², R³, R⁴, R⁵, R⁶, R⁷, R⁸, and R⁹ is not hydrogen;
R¹, R², and R³ are each independently selected from hydrogen, halo, and C₁₋₄ alkyl optionally substituted with from one to three halo substituents;
R⁴ is selected from hydrogen, halo, and C₁₋₄ alkyl optionally substituted with from one to three halo substituents, and
R⁵ is selected from hydrogen and C₁₋₄ alkyl, or
R⁴ and R⁵ are linked to form an ethane-1,2-diyl, a propane-1,3-diyl or a methane-1,1-diyloxy;
R⁶ is selected from hydrogen and C₁₋₄ alkyl;
R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has one or two heteroatoms as ring members in which one of the heteroatoms is nitrogen and another of the heteroatoms, if present, is independently selected from N, O, and S;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl.

Another aspect of the invention provides a compound which is selected from the group of compounds described in the examples and their pharmaceutically acceptable salts.

A further aspect of the invention provides a pharmaceutical composition which includes a compound of Formula 1 or a pharmaceutically acceptable salt thereof, or any one of the compounds or pharmaceutically acceptable salts defined in the preceding paragraph; and a pharmaceutically acceptable excipient.

An additional aspect of the invention provides a compound of Formula 1 or a pharmaceutically acceptable salt thereof, or any one of the compounds and pharmaceutically acceptable salts defined in the preceding paragraphs, for use as a medicament.

Another aspect of the invention provides a compound of Formula 1 or a pharmaceutically acceptable salt thereof, or any one of the compounds or pharmaceutically acceptable salts defined in the preceding paragraphs, for treatment of a disease, disorder or condition associated with PHD.

A further aspect of the invention provides a compound of Formula 1 or a pharmaceutically acceptable salt thereof, or any one of the compounds or pharmaceutically acceptable salts defined in the preceding paragraphs, for treatment of a disease, disorder or condition selected from cardiovascular disorders, metabolic disorders, hematological disorders, pulmonary disorders, kidney disorders, liver disorders, wound healing disorders, and cancer.

An additional aspect of the invention provides a compound of Formula 1 or a pharmaceutically acceptable salt thereof, or any one of the compounds or pharmaceutically acceptable salts defined in the preceding paragraphs, for treatment of a disease, disorder or condition selected from stroke, myocardial infarction, congestive heart failure, atherosclerosis, chronic venous insufficiency, cardiac cirrhosis, acute decompensated heart failure, heart failure following a heart attack, peripheral artery disease, occlusive artery disease, diabetes, hyperglycemia, insulin resistance, metabolic syndrome X, impaired glucose tolerance, non-alcoholic liver steatosis, anemia, chronic obstructive pulmonary disease, pulmonary embolism, pulmonary hypertension, mountain sickness, acute respiratory failure, interstitial lung disease, idiopathic pulmonary fibrosis, desquamative interstitial pneumonia, nonspecific interstitial pneumonia, cryptogenic organizing pneumonia, respiratory bronchiolitis-associated interstitial lung disease, acute interstitial pneumonia, lymphoid interstitial pneumonia, acute kidney failure, acute kidney injury, chronic kidney disease, renal ischemia reperfusion injury, hepatic ischemia reperfusion injury, diabetic foot ulcers, pressure ulcers, venous ulcers, arterial ulcers, epidermolysis bullosa, pemphigus, and Sjogren's Syndrome, and cancer.

Another aspect of the invention provides a use of a compound of Formula 1 or a pharmaceutically acceptable salt thereof, or any one of the compounds or pharmaceutically acceptable salts defined in the preceding paragraphs, for the manufacture of a medicament for the treatment of a disease, disorder or condition associated with PHD.

A further aspect of the invention provides a use of a compound of Formula 1 or a pharmaceutically acceptable salt thereof, or any one of the compounds or pharmaceutically acceptable salts defined in the preceding paragraphs, for the manufacture of a medicament for the treatment of a disease, disorder or condition selected from stroke, myocardial infarction, congestive heart failure, atherosclerosis, chronic venous insufficiency, cardiac cirrhosis, acute decompensated heart failure, heart failure following a heart attack, peripheral artery disease, occlusive artery disease, diabetes, hyperglycemia, insulin resistance, metabolic syndrome X, impaired glucose tolerance, non-alcoholic liver steatosis, anemia, chronic obstructive pulmonary disease, pulmonary embolism, pulmonary hypertension, mountain sickness, acute respiratory failure, interstitial lung disease, idiopathic pulmonary fibrosis, desquamative interstitial pneumonia, nonspecific interstitial pneumonia, cryptogenic organizing pneumonia, respiratory bronchiolitis-associated interstitial lung disease, acute interstitial pneumonia, lymphoid interstitial pneumonia, acute kidney failure, acute kidney injury, chronic kidney disease, renal ischemia reperfusion injury, hepatic ischemia reperfusion injury, diabetic foot ulcers, pressure ulcers, venous ulcers, arterial ulcers, epidermolysis bullosa, pemphigus, and Sjogren's Syndrome, and cancer.

An additional aspect of the invention provides a method of treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is associated with PHD.

Another aspect of the invention provides a method of treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is selected from cardiovascular disorders, metabolic disorders, hematological disorders, pulmonary disorders, kidney disorders, liver disorders, wound healing disorders, and cancer.

A further aspect of the invention provides a method of treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is selected from stroke, myocardial infarction, congestive heart failure, atherosclerosis, chronic venous insufficiency, cardiac cirrhosis, acute decompensated heart failure, heart failure following a heart attack, peripheral artery disease, occlusive artery disease, diabetes, hyperglycemia, insulin resistance, metabolic syndrome X, impaired glucose tolerance, non-alcoholic liver steatosis, anemia, chronic obstructive pulmonary disease, pulmonary embolism, pulmonary hypertension, mountain sickness, acute respiratory failure, interstitial lung disease, idiopathic pulmonary fibrosis, desquamative interstitial pneumonia, nonspecific interstitial pneumonia, cryptogenic organizing pneumonia, respiratory bronchiolitis-associated interstitial lung disease, acute interstitial pneumonia, lymphoid interstitial pneumonia, acute kidney failure, acute kidney injury, chronic kidney disease, renal ischemia reperfusion injury, hepatic ischemia reperfusion injury, diabetic foot ulcers, pressure ulcers, venous ulcers, arterial ulcers, epidermolysis bullosa, pemphigus, and Sjogren's Syndrome, and cancer.

An additional aspect of the invention provides a combination comprising a compound of Formula 1 or a pharmaceutically acceptable salt thereof, or any one of the compounds or pharmaceutically acceptable salts defined in the preceding paragraphs; and at least one additional pharmacologically active agent.

### DETAILED DESCRIPTION OF THE INVENTION

Unless otherwise indicated, this disclosure uses definitions provided below.

“Substituted,” when used in connection with a chemical substituent or moiety (e.g., a C₁₋₆ alkyl group), means that one or more hydrogen atoms of the substituent or moiety have been replaced with one or more non-hydrogen atoms or groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value or within +10 percent of the indicated value, whichever is greater.

“Alkyl” refers to straight chain and branched saturated hydrocarbon groups, generally having a specified number of carbon atoms (e.g., C₁₋₄ alkyl refers to an alkyl group having 1 to 4 (i.e., 1, 2, 3 or 4) carbon atoms, C₁₋₆ alkyl refers to an alkyl group having 1 to 6 carbon atoms, and so on). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkanediyl” refers to divalent alkyl groups, where alkyl is defined above, and generally having a specified number of carbon atoms (e.g., C₁₋₄ alkanediyl refers to an alkanediyl group having 1 to 4 (i.e., 1, 2, 3 or 4) carbon atoms, C₁₋₆ alkanediyl refers to an alkanediyl group having 1 to 6 carbon atoms, and so on). Examples of alkanediyl groups include methylene, ethane-1,1-diyl, ethane-1,2-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, isobutane-1,3-diyl, isobutane-1,1-diyl, isobutane-1,2-diyl, and the like.

“Alkenyl” refers to straight chain and branched hydrocarbon groups having one or more carbon-carbon double bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, 1-fluoroethyl, 1,1-difluoroethyl, 1-chloroethyl, 1,1-dichloroethyl, 1-fluoro-1-methylethyl, 1-chloro-1-methylethyl, and the like.

“Cycloalkyl” refers to saturated monocyclic and bicyclic hydrocarbon groups, generally having a specified number of carbon atoms that comprise the ring or rings (e.g., C₃₋₈ cycloalkyl refers to a cycloalkyl group having 3 to 8 carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). The cycloalkyl group may be attached through any ring atom unless such attachment would violate valence requirements, and where indicated, may optionally include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

“Cycloalkylidene” refers to divalent monocyclic cycloalkyl groups, where cycloalkyl is defined above, which are attached through a single carbon atom of the group, and generally having a specified number of carbon atoms that comprise the ring (e.g., C₃₋₆ cycloalkylidene refers to a cycloalkylidene group having 3 to 6 carbon atoms as ring members). Examples include cyclopropylidene, cyclobutylidene, cyclopentylidene, and cyclohexylidene.

“Cycloalkenyl” refers to partially unsaturated monocyclic and bicyclic hydrocarbon groups, generally having a specified number of carbon atoms that comprise the ring or rings. As with cycloalkyl groups, the bicyclic cycloalkenyl groups may include isolated, spiro, fused, or bridged rings. Similarly, the cycloalkenyl group may be attached through any ring atom, and where indicated, may optionally include one or more non-hydrogen substituents unless such attachment or substitution would violate valence requirements. Examples of cycloalkenyl groups include the partially unsaturated analogs of the cycloalkyl groups described above, such as cyclobutenyl (i.e., cyclobuten-1-yl and cyclobuten-3-yl), cyclopentenyl, cyclohexenyl, bicyclo[2.2.1]hept-2-enyl, and the like.

“Aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members). The group may be attached through any ring atom, and where indicated, may optionally include one or more non-hydrogen substituents unless such attachment or substitution would violate valence requirements. Examples of aryl groups include phenyl, biphenyl, cyclobutabenzenyl, indenyl, naphthalenyl, benzocycloheptanyl, biphenylenyl, fluorenyl, groups derived from cycloheptatriene cation, and the like.

“Arylene” refers to divalent aryl groups, where aryl is defined above. Examples of arylene groups include phenylene (i.e., benzene-1,2-diyl).

“Heterocycle” and “heterocyclyl” may be used interchangeably and refer to saturated or partially unsaturated monocyclic or bicyclic groups having ring atoms composed of carbon atoms and 1 to 4 heteroatoms independently selected from nitrogen, oxygen, and sulfur. Both the monocyclic and bicyclic groups generally have a specified number of carbon atoms in their ring or rings (e.g., C₂₋₆ heterocyclyl refers to a heterocyclyl group having 2 to 6 carbon atoms and 1 to 4 heteroatoms as ring members). As with bicyclic cycloalkyl groups, bicyclic heterocyclyl groups may include isolated rings, spiro rings, fused rings, and bridged rings. The heterocyclyl group may be attached through any ring atom, and where indicated, may optionally include one or more non-hydrogen substituents unless such attachment or substitution would violate valence requirements or result in a chemically unstable compound. Examples of heterocyclyl groups include oxiranyl, thiiranyl, aziridinyl (e.g., aziridin-1-yl and aziridin-2-yl), oxetanyl, thietanyl, azetidinyl, tetrahydrofuranyl, tetrahydrothienyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 3,6-dihydro-2H-pyranyl, 2H-pyranyl, 1,2-dihydropyridinyl, 1,2,3,4-tetrahydropyridinyl, 1,2,5,6-tetrahydropyridinyl, 1,6-dihydropyrimidinyl, 1,2,3,4-tetrahydropyrimidinyl, and 1,2-dihydropyrazolo[1,5-d][1,2,4]triazinyl.

“Heterocycle-diyl” refers to heterocyclyl groups which are attached through two ring atoms of the group, where heterocyclyl is defined above. They generally have a specified number of carbon atoms in their ring or rings (e.g., C₂₋₆ heterocycle-diyl refers to a heterocycle-diyl group having 2 to 6 carbon atoms and 1 to 4 heteroatoms as ring members). Examples of heterocycle-diyl groups include the multivalent analogs of the heterocycle groups described above, such as morpholine-3,4-diyl, pyrrolidine-1,2-diyl, 1-pyrrolidinyl-2-ylidene, 1-pyridinyl-2-ylidene, 1-(4H)-pyrazolyl-5-ylidene, 1-(3H)-imidazolyl-2-ylidene, 3-oxazolyl-2-ylidene, 1-piperidinyl-2-ylidene, 1-piperazinyl-6-ylidene, and the like.

“Heteroaromatic” and “heteroaryl” may be used interchangeably and refer to unsaturated monocyclic aromatic groups and to polycyclic groups having at least one aromatic ring, the groups having ring atoms composed of carbon atoms and 1 to 4 heteroatoms independently selected from nitrogen, oxygen, and sulfur. Both the monocyclic and polycyclic groups generally have a specified number of carbon atoms as ring members (e.g., C₁₋₉ heteroaryl refers to a heteroaryl group having 1 to 9 carbon atoms and 1 to 4 heteroatoms as ring members) and may include any bicyclic group in which any of the above-listed monocyclic heterocycles are fused to a benzene ring. The heteroaryl group may be attached through any ring atom (or ring atoms for fused rings), and where indicated, may optionally include one or more non-hydrogen substituents unless such attachment or substitution would violate valence requirements or result in a chemically unstable compound. For the purposes of this disclosure, 2-pyridone and 4-pyridone, 2-quinolone and 4-quinolone, and the like, are considered to be 2-oxo- and 4-oxo-substituted derivatives of the corresponding heteroaromatic group (pyridine, quinoline, and the like).

Examples of heteroaryl groups include monocyclic groups such as pyrrolyl (e.g., pyrrol-1-yl, pyrrol-2-yl, and pyrrol-3-yl), furanyl, thienyl, pyrazolyl, imidazolyl, isoxazolyl, oxazolyl, isothiazolyl, thiazolyl, 1,2,3-triazolyl, 1,3,4-triazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothienyl, benzo[c]thienyl, 1H-indolyl, 3H-indolyl, isoindolyl, 1H-isoindolyl, indolinyl, isoindolinyl, benzimidazolyl, 1H-indazolyl, 2H-indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, 1,2,3,4-tetrahydropyrido[2,3-b]pyrazinyl, 2,3-dihydrobenzo[b][1,4]dioxinyl, 3,4-dihydro-2H-pyrido[3,2-b][1,4]oxazinyl, 2,3-dihydro-1H-benzo[d]imidazolyl, benzo[d]thiazolyl, 2,3-dihydro-1H-pyrrolo[2,3-b]pyridinyl, [1,2,4]triazolo[1,5-a]pyridinyl, 2,3-dihydro-1H-imidazo[4,5-b]pyridinyl, tetrazolo[1,5-a]pyridinyl, 7H-pyrrolo[2,3-d]pyrimidinyl, pyrazolo[1,5-a]pyrimidinyl, imidazo[1,2-a]pyrimidinyl, 4,5-dihydro-1H-pyrazolo[3,4-d]pyrimidinyl, 2,3,6,7-tetrahydro-1H-purinyl, 5H-pyrrolo[2,3-b]pyrazinyl, imidazo[1,2-a]pyrazinyl, imidazo[1,2-b]pyridazinyl, and 4,5,6,7-tetrahydropyrazolo[1,5-a]pyrazinyl.

Other examples of include heteroaryl groups also include bicyclic groups 2,3-dihydrobenzofuranyl, 2-oxo-1,2,5,6,7,8-hexahydroquinolinyl, 4-oxo-4H-pyrido[1,2-a]pyrimidinyl, 5,6,7,8-tetrahydropyrazolo[5,1-b][1,3]oxazepinyl, 5,6-dihydro-4H-pyrrolo[1,2-b]pyrazolyl, 5-oxo-5H-thiazolo[3,2-a]pyrimidinyl, 6,7-dihydro-5H-cyclopenta[b]pyridinyl, 6,7-dihydro-5H-pyrazolo[5,1-b][1,3]oxazinyl, and pyrrolo[1,2-c]pyrimidinyl.

“Heteroarylene” refers to heteroaryl groups which are attached through two ring atoms of the group, where heteroaryl is defined above. They generally have a specified number of carbon atoms in their ring or rings (e.g., C₃₋₅ heteroarylene refers to a heteroarylene group having 3 to 5 carbon atoms and 1 to 4 heteroatoms as ring members). Examples of heteroarylene groups include the multivalent analogs of the heteroaryl groups described above, such as pyridine-2,3-diyl, pyridine-3,4-diyl, pyrazole-4,5-diyl, pyrazole-3,4-diyl, and the like.

“Oxo” refers to a double bonded oxygen (═O).

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Opposite enantiomer” refers to a molecule that is a non-superimposable mirror image of a reference molecule, which may be obtained by inverting all of the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomer” and “stereoisomers” of a compound with given stereochemical configuration refer to the opposite enantiomer of the compound and to any diastereoisomers, including geometrical isomers (Z/E) of the compound. For example, if a compound has S,R,Z stereochemical configuration, its stereoisomers would include its opposite enantiomer having R,S,Z configuration, and its diastereomers having S,S,Z configuration, R,R,Z configuration, S,R,E configuration, R,S,E configuration, S,S,E configuration, and R,R,E configuration. If the stereochemical configuration of a compound is not specified, then “stereoisomer” refers to any one of the possible stereochemical configurations of the compound.

“Substantially pure stereoisomer” and variants thereof refer to a sample containing a compound having a specific stereochemical configuration and which comprises at least about 95% of the sample.

“Pure stereoisomer” and variants thereof refer to a sample containing a compound having a specific stereochemical configuration and which comprises at least about 99.5% of the sample.

“Subject” refers to a mammal, including a human.

“Pharmaceutically acceptable” substances refer to those substances which are suitable for administration to subjects.

“Treating” refers to reversing, alleviating, inhibiting the progress of, or preventing a disease, disorder or condition to which such term applies, or to reversing, alleviating, inhibiting the progress of, or preventing one or more symptoms of such disease, disorder or condition.

“Treatment” refers to the act of “treating,” as defined immediately above.

“Drug,” “drug substance,” “active pharmaceutical ingredient,” and the like, refer to a compound (e.g., compounds of Formula 1, including subgeneric compounds and compounds specifically named in the specification) that may be used for treating a subject in need of treatment.

“Effective amount” of a drug, “therapeutically effective amount” of a drug, and the like, refer to the quantity of the drug that may be used for treating a subject and may depend on the weight and age of the subject and the route of administration, among other things.

“Excipient” refers to any diluent or vehicle for a drug.

“Pharmaceutical composition” refers to the combination of one or more drug substances and one or more excipients.

“Drug product,” “pharmaceutical dosage form,” “dosage form,” “final dosage form” and the like, refer to a pharmaceutical composition suitable for treating a subject in need of treatment and generally may be in the form of tablets, capsules, sachets containing powder or granules, liquid solutions or suspensions, patches, films, and the like.

“Condition associated with PHD” and similar phrases relate to a disease, disorder or condition in a subject for which inhibition of PHD may provide a therapeutic or prophylactic benefit.

The following abbreviations may be used in the specification: Ac (acetyl); ACN (acetonitrile); AIBN (azo-bis-isobutyronitrile); API (active pharmaceutical ingredient); aq (aqueous); BINAP (2,2′-bis(diphenylphosphino)-1,1′-binaphthyl); Boc (tert-butoxycarbonyl); Cbz (carbobenzyloxy); dba (dibenzylideneacetone); DCC (1,3-dicyclohexylcarbodiimide); DCE (1,1-dichloroethane); DCM (dichloromethane); DIPEA (N,N-diisopropylethylamine, Htinig's Base); DMA (N,N-dimethylacetamide); DMAP (4-dimethylaminopyridine); DME (1,2-dimethoxyethane); DMF (N,N-dimethylformamide); DMSO (dimethylsulfoxide); dppf (1,1′-bis(diphenylphosphino)ferrocene); DTT (dithiothreitol); EC₅₀ (effective concentration at half maximal response); EDA ethoxylated dodecyl alcohol, Brj®35); EDC (N-(3-dimethylaminopropyl)-N′-ethylcarbodiimide); EDTA (ethylenediaminetetraacetic acid); ee (enantiomeric excess); eq (equivalents); Et (ethyl); Et₃N (triethyl-amine); EtOAc (ethyl acetate); EtOH (ethanol); HATU (2-(3H-[1,2,3]triazolo[4,5-b]pyridin-3-yl)-1,1,3,3-tetramethyluronium hexafluorophosphate(V)); HBTU (2-(1H-benzotriazol-1-yl)-1,1,3,3-tetramethyluronium hexafluorophosphate); HEPES (4-(2-hydroxyethyl)piperazine-1-ethanesulfonic acid); AcOH (acetic acid); HOBt (1H-benzo[d][1,2,3]triazol-1-ol); IC₅₀ (concentration at 50% inhibition); IPA (isopropanol); IPAc (isopropyl acetate); IPE (isopropylether); LDA (lithium diisopropylamide); LiHMDS (lithium bis(trimethylsilyl)amide); mCPBA (m-chloroperoxybenzoic acid); Me (methyl); MeOH (methanol); MTBE (methyl tert-butyl ether); mp (melting point); NaOt-Bu (sodium tertiary butoxide); NMM (N-methylmorpholine); NMP (N-methyl-pyrrolidone); OTf (triflate); PE (petroleum ether); Ph (phenyl); pEC₅₀ (−log₁₀(EC₅₀), where EC₅₀ is given in molar (M) units); pIC₅₀ (−log₁₀(IC₅₀), where IC₅₀ is given in molar (M) units); Pr (propyl); c-Pr (cyclopropyl), i-Pr (isopropyl); PTFE (polytetrafluoroethylene); RT (room temperature, approximately 20° C. to 25° C.); T3P (2,4,6-tripropyl-1,3,5,2,4,6-trioxatriphosphinane 2,4,6-trioxide); TCEP (tris(2-carboxyethyl)phosphine); TFA (trifluoroacetic acid); TFAA (2,2,2-trifluoroacetic anhydride); THF (tetrahydrofuran); TMEDA (N¹,N¹,N²,N²-tetramethylethane-1,2-diamine); TMS (trimethylsilyl); and Tris buffer (2-amino-2-hydroxymethyl-propane-1,3-diol buffer).

As described, below, this disclosure concerns compounds of Formula 1 and their pharmaceutically acceptable salts. This disclosure also concerns materials and methods for preparing compounds of Formula 1, pharmaceutical compositions which contain them, and the use of compounds of Formula 1 and their pharmaceutically acceptable salts (optionally in combination with other pharmacologically active agents) for treating diseases, disorders or conditions associated with PHD.

The compounds of Formula 1 include those in which (1):
X¹ is selected from N and CR¹, and
X² is selected from N and CR², provided:
   (a) no more than one of X¹ and X² is N, and
   (b) when R⁴ and R⁵ are linked to form an ethane-1,2-diyl, a propane-1,3-diyl or a methane-1,1-diyloxy, then X¹ is CR¹ and X² is CR², and
   (c) when X¹ is CR¹ and X² is CR², then at least one of R¹, R², R³, R⁴, R⁵, R⁶, R⁷, R⁸, and R⁹ is not hydrogen;
R¹, R², and R³ are each independently selected from hydrogen, halo, and C₁₋₄ alkyl optionally substituted with from one to three halo substituents;
R⁴ is selected from hydrogen, halo, and C₁₋₄ alkyl optionally substituted with from one to three halo substituents, and
R⁵ is selected from hydrogen and C₁₋₄ alkyl, or
R⁴ and R⁵ are linked to form an ethane-1,2-diyl, a propane-1,3-diyl or a methane-1,1-diyloxy;
R⁶ is selected from hydrogen and C₁₋₄ alkyl;
R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has one or two heteroatoms as ring members in which one of the heteroatoms is nitrogen and another of the heteroatoms, if present, is independently selected from N, O, and S;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl.

In addition to the specific compounds in the examples, the compounds of Formula 1 include those in which:
(2) X¹ is CR¹;
(3) X² is CR²; or
(4) X¹ is CR¹ and X² is CR²;

In addition, or as an alternative, to one of embodiments (1) through (4) in the preceding paragraphs, compounds of Formula 1 include those in which:
(5) R¹ is selected from hydrogen, halo, and methyl;
(6) R¹ is selected from hydrogen, fluoro, chloro, and methyl;
(7) R¹ is selected from hydrogen, fluoro, and methyl; or
(8) R¹ is selected from hydrogen and methyl.

In addition, or as an alternative, to one of embodiments (1) to (8) in the preceding paragraphs, compounds of Formula 1 include those in which:
(9) R² is selected from hydrogen, halo, and methyl;
(10) R² is selected from hydrogen, fluoro, and methyl; or
(11) R² is selected from hydrogen and methyl.

In addition, or as an alternative, to one of embodiments (1) to (11) in the preceding paragraphs, compounds of Formula 1 include those in which:
(12) R³ is selected from hydrogen, halo, and methyl;
(13) R³ is selected from hydrogen, fluoro, and methyl; or
(14) R³ is selected from hydrogen and methyl.

In addition, or as an alternative, to one of embodiments (1) through (14) in the preceding paragraphs, compounds of Formula 1 include those in which:
(15) R⁴ is selected from hydrogen, halo, and methyl;
(16) R⁴ is selected from hydrogen, fluoro, chloro, and methyl;
(17) R⁴ is selected from hydrogen, fluoro, and methyl; or
(18) R⁴ is selected from hydrogen and methyl.

In addition, or as an alternative, to one of embodiments (1) to (18) in the preceding paragraphs, compounds of Formula 1 include those in which:
(19) R⁵ is selected from hydrogen and methyl; or
(20) R⁵ is hydrogen.

In addition, or as an alternative, to one of embodiments (1) to (20) in the preceding paragraphs, compounds of Formula 1 include those in which:
(21) R⁶ is selected from hydrogen and methyl; or
(22) R⁶ is hydrogen.

In addition, or as an alternative, to one of embodiments (5) to (14) in the preceding paragraphs, compounds of Formula 1 include those in which:
(23) R⁴ and R⁵ are linked to form an ethane-1,2-diyl or a propane-1,3-diyl; or
(24) R⁴ and R⁵ are linked to form an ethane-1,2-diyl.

In addition, or as an alternative, to one of embodiments (1) to (24) in the preceding paragraphs, compounds of Formula 1 include those in which:
(25) R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has 5 or 6 ring members and one or two heteroatoms as ring members in which one of the heteroatoms is nitrogen and another of the heteroatoms, if present, is independently selected from N, O, and S;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl;
(26) R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has 5 or 6 ring members and one or two heteroatoms as ring members, each of which is nitrogen;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl;
(27) R⁷, R⁸, and R⁹ are each independently selected from hydrogen, halo, cyano, —N(R^{a})R^{b}, —C(O)N(R^{a})R^{b}, —OR⁰, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₄₋₅ heterocyclyl optionally substituted with C₁₋₄ alkyl, wherein the C₁₋₄ alkyl substituent is optionally substituted with from one to three halo substituents, and the C₄₋₅ heterocyclyl moiety has 6 ring members and one or two heteroatoms as ring members in which one of the heteroatoms is nitrogen and another of the heteroatoms, if present, is independently selected from N, O, and S;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl;
(28) R⁷, R⁸, and R⁹ are each independently selected from hydrogen, halo, cyano, —N(R^{a})R^{b}, —C(O)N(R^{a})R^{b}, —OR⁰, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₄₋₅ heterocyclyl optionally substituted with C₁₋₄ alkyl, wherein the C₁₋₄ alkyl substituent is optionally substituted with from one to three halo substituents, and the C₄₋₅ heterocyclyl moiety has 6 ring members and one or two heteroatoms as ring members, each of which is nitrogen;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl;
(29) R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has one or two heteroatoms as ring members, each of which is nitrogen;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl; or
(30) R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
   R^{a} and R^{b} are each independently selected from hydrogen and C₁₋₄ alkyl optionally substituted with from one to three substituents independently selected from halo and —OR^{d}, or
   R^{a} and R^{b} together with the nitrogen atom to which they are attached form a piperazine-1-yl optionally substituted with C₁₋₄ alkyl, wherein the C₁₋₄ alkyl substituent is optionally substituted with from one to three halo substituents;
   R^{c} is selected from hydrogen and C₁₋₄ alkyl optionally substituted with from one to three substituents independently selected from halo and —OR^{d}; and
   R^{d} is selected from hydrogen and C₁₋₄ alkyl.

In addition, or as an alternative, to one of embodiments (1) to (30) in the preceding paragraphs, compounds of Formula 1 include those in which:
(31) R⁷ is selected from hydrogen, halo, cyano, and C₁₋₄ alkyl;
(32) R⁷ is selected from hydrogen, halo, cyano, and methyl;
(33) R⁷ is selected from hydrogen, halo, and methyl;
(34) R⁷ is selected from hydrogen, fluoro, chloro, and methyl; or
(35) R⁷ is hydrogen.

In addition, or as an alternative, to one of embodiments (1) to (35) in the preceding paragraphs, compounds of Formula 1 include those in which:
(36) R⁸ is selected from hydrogen, halo, cyano, —N(R^{a})R^{b}, —C(O)N(R^{a})R^{b}, —OR^{c}, methyl, and —CF₃;
(37) R⁸ is selected from hydrogen, fluoro, chloro, cyano, —N(R^{a})R^{b}, —C(O)N(R^{a})R^{b}, —OCH₃, methyl, and —CF₃;
(38) R⁸ is selected from hydrogen, fluoro, chloro, cyano, —N(H)R^{b}, —C(O)N(R^{a})R^{b}, —OCH₃, methyl, and —CF₃;
(39) R⁸ is selected from hydrogen, fluoro, chloro, cyano, —N(H)R^{b}, —OCH₃, methyl, and —CF₃;
(40) R⁸ is selected from hydrogen, fluoro, chloro, cyano, —N(H)CH₂OCH₃, —N(H)CH₂CH₂OCH₃, —N(H)CH₂CH₂CH₂OCH₃, —OCH₃, methyl, and —CF₃; or
(41) R⁸ is selected from hydrogen, fluoro, chloro, cyano, —N(H)CH₂CH₂CH₂OCH₃, —OCH₃, methyl, and —CF₃.

In addition, or as an alternative, to one of embodiments (1) to (41) in the preceding paragraphs, compounds of Formula 1 include those in which:
(42) R⁹ is selected from hydrogen, halo, cyano, and C₁₋₄ alkyl;
(43) R⁹ is selected from hydrogen, halo, cyano, and methyl;
(44) R⁹ is selected from hydrogen, halo, and methyl;
(45) R⁹ is selected from hydrogen, fluoro, chloro, and methyl; or
(46) R⁹ is hydrogen.

Compounds of Formula 1 include embodiments (1) through (46) described in the preceding paragraphs and final compounds specifically named in the examples (except for comparative examples) and may exist as salts, complexes, solvates, hydrates, and liquid crystals. Likewise, compounds of Formula 1 that are salts may exist as complexes, solvates, hydrates, and liquid crystals.

Compounds of Formula 1 may form pharmaceutically acceptable complexes, salts, solvates and hydrates. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound of Formula 1 may be reacted with an appropriate acid or base to give the desired salt. Alternatively, a precursor of the compound of Formula 1 may be reacted with an acid or base to remove an acid- or base-labile protecting group or to open a lactone or lactam group of the precursor. Additionally, a salt of the compound of Formula 1 may be converted to another salt (or free form) through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

Compounds of Formula 1 may exist in a continuum of solid states ranging from fully amorphous to fully crystalline. The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Compounds of Formula 1 may also exist in unsolvated and solvated forms. The term “solvate” describes a molecular complex comprising the compound and one or more pharmaceutically acceptable solvent molecules (e.g., ethanol). The term “hydrate” is a solvate in which the solvent is water. Pharmaceutically acceptable solvates include those in which the solvent may be isotopically substituted (e.g., D₂O, acetone-d₆, DMSO-d₆).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

Compounds of Formula 1 may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. (2004) 17:1889-1896. For a general review of multi-component complexes, see J. K. Haleblian, J. Pharm. Sci. (1975) 64(8):1269-88.

When subjected to suitable conditions, compounds of Formula 1 may exist in a mesomorphic state (mesophase or liquid crystal). The mesomorphic state lies between the true crystalline state and the true liquid state (either melt or solution). Mesomorphism arising as the result of a change in temperature is described as “thermotropic” and mesomorphism resulting from the addition of a second component, such as water or another solvent, is described as “lyotropic.” Compounds that have the potential to form lyotropic mesophases are described as “amphiphilic” and include molecules which possess a polar ionic moiety (e.g., —COO⁻Na⁺, —COO⁻K⁺, —SO₃⁻Na⁺) or polar non-ionic moiety (such as —N⁻N⁺(CH₃)₃). See, e.g., N. H. Hartshome and A. Stuart, Crystals and the Polarizing Microscope (4th ed, 1970).

Each compound of Formula 1 may exist as polymorphs, stereoisomers, tautomers, or some combination thereof, may be isotopically-labeled, may result from the administration of a prodrug, or form a metabolite following administration.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of compounds of Formula 1 having carboxylic acid, hydroxy, or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of Formula 1 having methyl, alkoxy, tertiary amino, secondary amino, phenyl, and amide groups, respectively.

Compounds of Formula 1 may exist as stereoisomers that result from the presence of one or more stereogenic centers, one or more double bonds, or both. The stereoisomers may be pure, substantially pure, or mixtures. Such stereoisomers may also result from acid addition or base salts in which the counter-ion is optically active, for example, when the counter-ion is D-lactate or L-lysine.

Compounds of Formula 1 may exist as tautomers, which are isomers resulting from tautomerization. Tautomeric isomerism includes, for example, imine-enamine, keto-enol, oxime-nitroso, and amide-imidic acid tautomerism.

Compounds of Formula 1 may exhibit more than one type of isomerism.

Geometrical (cis/trans) isomers may be separated by conventional techniques such as chromatography and fractional crystallization.

Conventional techniques for preparing or isolating a compound having a specific stereochemical configuration include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of Formula 1 contains an acidic or basic moiety, an acid or base such as tartaric acid or 1-phenylethylamine. The resulting diastereomeric mixture may be separated by chromatography, fractional crystallization, etc., and the appropriate diastereoisomer converted to the compound having the requisite stereochemical configuration. For a further discussion of techniques for separating stereoisomers, see E. L. Eliel and S. H. Wilen, Stereochemistry of Organic Compounds (1994).

Compounds of Formula 1 may possess isotopic variations, in which at least one atom is replaced by an atom having the same atomic number, but an atomic mass different from the atomic mass usually found in nature. Isotopes suitable for inclusion in compounds of Formula 1 include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

The compounds of Formula 1 may be prepared using the techniques described below. Some of the schemes and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in a number of treatises, including Richard Larock, Comprehensive Organic Transformations (1999), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a di-acid, etc.). In addition, in some instances, reaction intermediates may be used in subsequent steps without isolation or purification (i.e., in situ).

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry (1999) and P. Kocienski, Protective Groups (2000).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure and claims to a stoichiometric range, a temperature range, a pH range, etc., whether or not expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane, cyclohexane, methylcyclohexane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., hexamethylphosphoric triamide).

In the schemes, below, substituent identifiers (e.g., X¹, X², R³, R⁴, R⁵, R⁶, etc.) are as defined above for Formula 1. As mentioned earlier, however, some of the starting materials and intermediates may include protecting groups, which are removed prior to the final product. In such cases, the substituent identifier refers to moieties defined in Formula 1 and to those moieties with appropriate protecting groups. For example, a starting material or intermediate in the schemes may include an R⁷ substituent having a potentially reactive amine. In such cases, R⁷ would include the moiety with or without, say, a Boc or Cbz group attached to the amine.

Scheme A shows a general method for preparing compounds of Formula 1. According to the method, a 3-hydroxy picolinic acid derivative (A1, R¹⁰=hydrogen or protecting group such as methyl, benzyl, etc.) is reacted with an amine (A2) to give an amide (A3). Step 1 may be carried out using standard amide coupling agents, such as HATU, DCC, EDC hydrochloride, T3P, and 2-chloro-1-methylpyridin-1-ium iodide, in the presence of a non-nucleophilic base (e.g., Et₃N, DIPEA) and one or more compatible polar solvents (e.g. DCM, DMA, DMF, THF). The amide coupling may be carried out at temperatures which range from room temperature to about 80° C. HOBt may be used to facilitate the reaction. When R¹⁰ is non-hydrogen, the hydroxy group is deprotected (Step 2) to give the compound of Formula 1. For example, when R¹⁰ is methyl, the amide (A3) may be reacted with LiCl in DMA at elevated temperature (e.g., 60-80° C.) to give the compound of Formula 1. Similarly, when R¹⁰=benzyl, the amide (A3) may be reacted with H₂ in the presence of a suitable catalyst (e.g., Pd(OH)₂/C) and solvent (e.g., THF, MeOH, EtOH, IPA, etc.) at room temperature to give the compound of Formula 1. When R¹⁰ is hydrogen, the amide (A3) corresponds to the compound of Formula 1 and Step 2 is unnecessary.

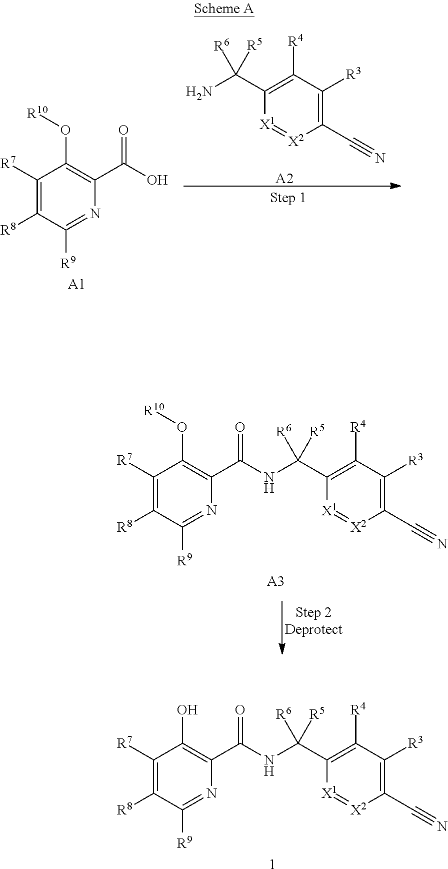

Scheme B shows an alternative method for preparing compounds of Formula 1. According to the method, a 3-halo picolinic acid derivative (B1, X³=fluoro, chloro, bromo) is reacted with an amine (A2) to give an aryl halide (B2). Step 1 may be carried out using standard amide coupling reagents and conditions noted above for Scheme A. The aryl halide (B2) is then reacted with NaOCH₃ in MeOH and an optional co-solvent (e.g. ACN) to give a 3-methoxy picolinic acid derivative (A3) which is subsequently treated with LiCl/DMA to give the compound of Formula 1.

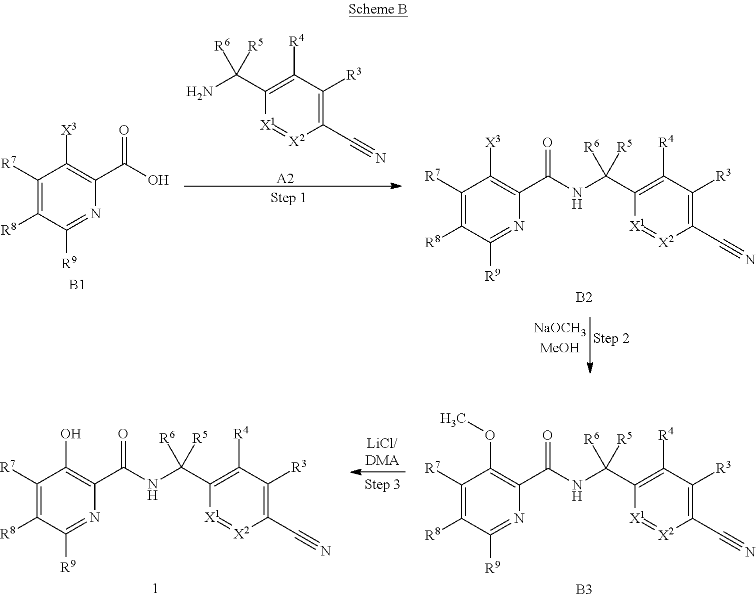

The methods depicted in the schemes may be varied as desired. For example, protecting groups may be added or removed and products (including intermediates) may be further elaborated via, for example, alkylation, acylation, hydrolysis, oxidation, reduction, amidation, sulfonation, alkynation, and the like to give the desired final product. Furthermore, any intermediate or final product which comprises mixture of stereoisomers may be optionally purified by chiral column chromatography (e.g., supercritical fluid chromatography) or by derivatization with optically-pure reagents as described above to give a desired stereoisomer.

Compounds of Formula 1, which include compounds named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

Compounds of Formula 1 may be administered alone or in combination with one another or with one or more pharmacologically active compounds which are different than the compounds of Formula 1. Generally, one or more of these compounds are administered as a pharmaceutical composition (a formulation) in association with one or more pharmaceutically acceptable excipients. The choice of excipients depends on the particular mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Compounds of Formula 1 may be administered orally. Oral administration may involve swallowing in which case the compound enters the bloodstream via the gastrointestinal tract. Alternatively or additionally, oral administration may involve mucosal administration (e.g., buccal, sublingual, supralingual administration) such that the compound enters the bloodstream through the oral mucosa.

Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, e.g., from gelatin or hydroxypropylmethylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

Compounds of Formula 1 may also be used in fast-dissolving, fast-disintegrating dosage forms such as those described in Liang and Chen, Expert Opinion in Therapeutic Patents (2001) 11(6):981-986.

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate.

Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet.

Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function.

In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film.

Film dosage forms are typically prepared by evaporative drying of thin aqueous films coated onto a peelable backing support or paper, which may carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14.

Compounds of Formula 1 may also be administered directly into the blood stream, muscle, or an internal organ of the subject. Suitable techniques for parenteral administration include intravenous, intraarterial, intraperitoneal, intrathecal, intraventricular, intraurethral, intrastemal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, compounds of Formula 1 may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, compounds of Formula 1 may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

Compounds of Formula 1 may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999).

Other means of topical administration include delivery by electroporation, iontophoresis, phonophoresis, sonophoresis and microneedle or needle-free (e.g. Powderject™ and Bioject™) injection. Formulations for topical administration may be formulated to be immediate or modified release as described above.

Compounds of Formula 1 may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more compounds of Formula 1, propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

Compounds of Formula 1 may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g. absorbable gel sponges, collagen), non-biodegradable implants (e.g. silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

To improve their solubility, dissolution rate, taste-masking, bioavailability, or stability, compounds of Formula 1 may be combined with soluble macromolecular entities, including cyclodextrin and its derivatives and polyethylene glycol-containing polymers. For example, API-cyclodextrin complexes are generally useful for most dosage forms and routes of administration. Both inclusion and non-inclusion complexes may be used. As an alternative to direct complexation with the API, the cyclodextrin may be used as an auxiliary additive, i.e. as a carrier, diluent, or solubilizer. Alpha-, beta- and gamma-cyclodextrins are commonly used for these purposes. See, e.g., WO 91/11172, WO 94/02518, and WO 98/55148.

As noted above, one or more compounds of Formula 1, including final compounds specifically named in examples, and their pharmaceutically active complexes, salts, solvates and hydrates, may be combined with each other or with one or more other active pharmaceutically active compounds to treat various diseases, conditions and disorders. In such cases, the active compounds may be combined in a single dosage form as described above or may be provided in the form of a kit which is suitable for coadministration of the compositions. The kit comprises (1) two or more different pharmaceutical compositions, at least one of which contains a compound of Formula 1; and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., pediatric patient) whose mass falls outside of this mass range.

The compounds of Formula 1 may be used to treat diseases, disorders, and conditions for which inhibition of PHD is indicated. As indicated above, inhibition of PHD may increase the stability and/or activity and/or level of hypoxia-inducible factor (HIF). As such, compounds that inhibit PHD may be useful for treating various diseases, disorders, and conditions where activation of HIF provides a therapeutic or prophylactic benefit, including diseases, disorders, and conditions involving hypoxia or ischemia. Such diseases, disorders, and conditions may include cardiovascular disorders, metabolic disorders, hematological disorders, pulmonary disorders, kidney disorders, liver disorders, wound healing disorders, and cancer, among others.

The compounds of Formula 1 may be used to treat cardiovascular diseases, disorders and conditions, including stroke; myocardial infarction, including acute myocardial infarction; congestive heart failure; atherosclerosis; chronic venous insufficiency; cardiac cirrhosis; acute decompensated heart failure; heart failure following a heart attack; peripheral artery disease; and occlusive artery disease.

The compounds of Formula 1 may be used to treat metabolic diseases, disorders and conditions, including diabetes, hyperglycemia, insulin resistance, metabolic syndrome X, impaired glucose tolerance, and non-alcoholic liver steatosis.

The compounds of Formula 1 may be used to treat hematological diseases, disorders and conditions, including anemia, which specifically includes, but is not limited to chemotherapy-induced anemia, such as treatment with antiviral drug regimens for HIV and hepatitis; anemia associated with chronic disease; anemia associated with cancer, including anemia resulting from treatment for cancer; anemia associated with chronic immune disorders, such as rheumatoid arthritis, inflammatory bowel disease, and lupus; anemia associated with menstruation, iron processing deficiencies, acute or chronic kidney disease, infections, inflammation, irradiation, toxins, diabetes, and infection due to, e.g., virus, bacteria, and/or parasites; anemia associated with blood loss due to, e.g., trauma, stomach ulcers, duodenal ulcers, hemorrhoids, cancer of the stomach or large intestine, injury, and surgical procedures; anemias associated with bone marrow failure or decreased bone marrow function; microcytic anemia; hypochromic anemia; sideroblastic anemia, and the like.

The compounds of Formula 1 may be used to treat pulmonary diseases, disorders and conditions, including chronic obstructive pulmonary disease (COPD); pulmonary embolism; pulmonary hypertension; mountain sickness; acute respiratory failure; interstitial lung diseases (ILD) including idiopathic ILD, such as idiopathic pulmonary fibrosis, desquamative interstitial pneumonia, nonspecific interstitial pneumonia, cryptogenic organizing pneumonia, respiratory bronchiolitis-associated interstitial lung disease, acute interstitial pneumonia, and lymphoid interstitial pneumonia.

The compounds of Formula 1 may be used to treat kidney diseases, disorders and conditions, including acute kidney failure; acute kidney injury; chronic kidney disease; and renal ischemia reperfusion injury.

The compounds of Formula 1 may be used to treat liver diseases, disorders and conditions, including hepatic ischemia reperfusion injury.

The compounds of Formula 1 may be used to treat wound healing diseases, disorders, and conditions, including diabetic foot ulcers, pressure ulcers, venous ulcers, arterial ulcers, epidermolysis bullosa (both genetic and acquired), pemphigus, and Sjogren's Syndrome.

The compounds of Formula 1 may be used to treat cancer, including leukemia (e.g., chronic myelogenous leukemia and chronic lymphocytic leukemia); breast cancer; genitourinary cancer; skin cancer; bone cancer; prostate cancer; liver cancer; brain cancer; cancer of the larynx, gall bladder, rectum, parathyroid, thyroid, adrenal, neural tissue, bladder, head, neck, stomach, bronchi, and kidneys; basal cell carcinoma, squamous cell carcinoma, metastatic skin carcinoma, osteosarcoma, Ewing's sarcoma, reticulum cell sarcoma, and Kaposi's sarcoma; myeloma, giant cell tumor, islet cell tumor, acute and chronic lymphocytic and granulocytic tumors; hairy-cell tumor, adenoma, medullary carcinoma, pheochromocytoma, mucosal neuromas, intestinal ganglioneuromas, hyperplastic corneal nerve tumor, marfanoid habitus tumor, Wilms' tumor, seminoma, ovarian tumor, leiomyomater tumor, cervical dysplasia, neuroblastoma, retinoblastoma, myelodysplastic syndrome, rhabdomyosarcoma, astrocytoma, non-Hodgkin's lymphoma, malignant hypercalcemia, polycythermia vera, adenocarcinoma, glioblastoma multiforma, glioma, lymphomas, and malignant melanomas.

The claimed and disclosed compounds may be combined with one or more other pharmacologically active compounds or therapies to treat one or more diseases, disorders or conditions associated with PHD. Such combinations may offer significant therapeutic advantages, including fewer side effects, improved ability to treat underserved patient populations, or synergistic activity. Compounds of Formula 1, which include compounds specifically named in examples, and their pharmaceutically acceptable complexes, salts, solvates and hydrates, may be administered simultaneously, sequentially or separately in combination with one or more compounds or therapies for cardiovascular disorders, metabolic disorders, hematological disorders, pulmonary disorders, kidney disorders, liver disorders, wound healing disorders, and cancer, among others.

For example, the compounds of Formula 1 may be combined with one or more cardiovascular agents such as calcium channel blockers, including amlodipine, clevidipine, diltiazem, felodipine, isradipine, nicardipine, nifedipine, nisoldipine, and verapamil; statins, including atorvastatin, fluvastatin, lovastatin, pravastatin, rosuvastatin, simvastatin, and pitavastatin; fibrates, including gemfibrozil and fenofibrate; beta-blockers, including acebutolol, atenolol, betaxolol, bisoprolol, carvedilol, esmolol, labetalo metoprolol, nadolol, nebivolol, penbutolol, propranolol, sotalol, and timolol; ACE inhibitors, including benazepril, captopril, enalapril, fosinopril, Lisinopril, moexipril, perindopril, quinapril, ramipril, and trandolapril; and platelet aggregation inhibitors, including aspirin, cangrelor, clopidogrel, cilostazol, dipyridamole, prasugrel, and ticagrelor.

The compounds of Formula 1 may be combined with one or more agents for treating metabolic disorders. These agents include pancreatic lipase inhibitors (e.g., orlistat); insulin; insulin sensitizers, including biguanides (e.g., buformin, metformin, and phenformin) and glitazones (e.g., pioglitazone and rosiglitazone); insulin secretagogues, including sulfonylureas (e.g., acetohexamide, chlorpropamide, tolazamide, tolbutamide, gliclazide, glimepiride, glipizide, and glyburide), and meglitinides (e.g., nateglinide and repaglinide); alpha-glucosidase inhibitors (e.g., acarbose and miglitol); glucagon-like peptide analogs and agonists (e.g., exenatide, liraglutide, and taspoglutide); dipeptidyl peptidase-4 inhibitors (e.g., alogliptin, linagliptin, saxagliptin, sitagliptin, and vildagliptin); and amylin analogs (e.g., pramlinitide).

The compounds of Formula 1 may be combined with one or more therapies or agents for treating wound healing disorders, including anti-inflammatory agents, analgesics, antipruritics, and anti-infectives. Examples of anti-inflammatory agents include nonsteroidal anti-inflammatory drugs (NSAIDs) and corticosteroids. Representative NSAIDs include apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac. Representative corticosteroids include betamethasone, cortisone acetate, dexamethasone, hydrocortisone, methylprednisolone, prednisolone, and prednisone. Representative analgesics include acetaminophen and morphine sulfate, as well as codeine, hydrocodone, oxycodone, propoxyphene, and tramadol, all with or without acetaminophen. Representative antipruritics for systemic use include cyproheptadine, diphenhydramine, gabapentin, hydroxyzine, and ondansetron. Representative antipruritics for topical use include ammonium lactate, benzocaine, calamine, capsaicin, clioquinol, crotamiton, diphenhydramine, doxepin, hydrocortisone, lidocaine, menthol, methyl salicylate, and pramoxine.

Example anti-infective agents may include antibacterials, antifungals, and antivirals. Representative antibacterials include aminoglycosides, such as amikacin, gentamicin, kanamycin, neomycin, paromomycin, and tobramycin; carbapenems, such as doripenem, ertapenem, imipenem, and meropenem; cephalosporins, including combinations with beta-lactamase inhibitors such as ceftazidime/avibactam and ceftolozane/tazobactam; first-generation cephalosporins, such as cefadroxil, cefazolin, cephalexin, and cephradine; second-generation cephalosporins, such as cefotetan, cefprozil, cefuroxime, efoxitin, and loracarbef; third-generation cephalosporins, such as cefdinir, cefditoren, cefixime, cefoperazone, cefotaxime, cefpodoxime, ceftazidime, ceftibuten, ceftizoxime, and ceftriaxone; and fourth- and next-generation cephalosporins, such as cefepime and ceftaroline; glycopeptide antibiotics, such as dalbavancin, oritavancin, telavancin, and vancomycin; glycylcyclines, such as tigecycline; lincomycin and its derivatives, such as clindamycin; macrolides, such as azithromycin, clarithromycin, erythromycin, and fidaxomicin, and macrolide derivatives, including ketolides such as telithromycin; oxazolidinone antibiotics, such as linezolid and tedizolid; penicillins, including aminopenicillins, such as amoxicillin and ampicillin; antipseudomonal penicillins, such as carbenicillin, piperacillin, and ticarcillin; penicillins with beta-lactamase inhibitors such as amoxicillin/clavulanate, ampicillin/sulbactam, piperacillin/tazobactam, and ticarcillin/clavulanate; natural penicillins, such as penicillin G benzathine, penicillin V potassium, and procaine penicillin; penicillinase resistant penicillins, such as dicloxacillin, nafcillin, and oxacillin; quinolones, such as cinoxacin, ciprofloxacin, delafloxacin, gatifloxacin, gemifloxacin, levofloxacin, lomefloxacin, moxifloxacin, nalidixic acid, norfloxacin, ofloxacin, sparfloxacin, and trovafloxacin; sulfonamides, such as sulfamethoxazole/trimethoprim and sulfisoxazole; tetracycline and its derivatives, such as demeclocycline, doxycycline, doxycycline/omega-3 polyunsaturated fatty acids, doxycycline/salicylic acid, minocycline, and oxytetracycline. Other representative antibacterials include atovaquone, aztreonam, bacitracin, chloramphenicol, colistimethate, dalfopristin/quinupristin, daptomycin, erythromycin/sulfisoxazole, fosfomycin, metronidazole, pentamidine, rifaximin, spectinomycin, and trimetrexate.

Representative antifungals include azole antifungals, such as clotrimazole, fluconazole, isavuconazonium, itraconazole, ketoconazole, miconazole, posaconazole, and voriconazole; echinocandins, such as anidulafungin, caspofungin, and micafungin; and polyenes, such as amphotericin B, amphotericin B cholesteryl sulfate, amphotericin B lipid complex, and nystatin. Other representative antifungals include flucytosine, griseofulvin, and terbinafine.

Representative antiviral agents include purine nucleosides, such as acyclovir, cidofovir, famciclovir, ganciclovir, ribavirin, valacyclovir, and valganciclovir.

In addition to anti-inflammatory agents, analgesics, antipruritics, and anti-infectives, the compounds of Formula 1 may be combined with cell or gene therapies for healing wounds.

The compounds of Formula 1 may also be combined with one or more compounds or therapies for treating cancer. These include chemotherapeutic agents (i.e., cytotoxic or antineoplastic agents) such as alkylating agents, antibiotics, antimetabolic agents, plant-derived agents, and topoisomerase inhibitors, as well as molecularly targeted drugs which block the growth and spread of cancer by interfering with specific molecules involved in tumor growth and progression. Molecularly targeted drugs include both small molecules and biologics.

Representative alkylating agents include bischloroethylamines (nitrogen mustards) including chlorambucil, cyclophosphamide, ifosfamide, mechlorethamine, melphalan, and uracil mustard); aziridines, including thiotepa; alkyl alkone sulfonates, including busulfan; nitrosoureas, including carmustine, lomustine, and streptozocin; nonclassical alkylating agents, including altretamine, dacarbazine, and procarbazine; and platinum compounds, including carboplatin, cisplatin, nedaplatin, oxaliplatin, satraplatin, and triplatin tetranitrate.

Representative antibiotic agents include anthracyclines, including aclarubicin, amrubicin, daunorubicin, doxorubicin, epirubicin, idarubicin, pirarubicin, valrubicin, and zorubicin; anthracenediones, including mitoxantrone and pixantrone; and Streptomyces, including actinomycin, bleomycin, dactinomycin, mitomycin C, and plicamycin.

Representative antimetabolic agents include dihydrofolate reductase inhibitors, including aminopterin, methotrexate, and pemetrexed; hymidylate synthase inhibitors, including raltitrexed and pemetrexed; folinic acid, including leucovorin; adenosine deaminase inhibitors, including pentostatin; halogenated/ribonucleotide reductase inhibitors, including cladribine, clofarabine, and fludarabine; thiopurines, including thioguanine and mercaptopurine; thymidylate synthase inhibitors, including fluorouracil, capecitabine, tegafur, carmofur, and floxuridine; DNA polymerase inhibitors, including cytarabine; ribonucleotide reductase inhibitors, including gemcitabine; hypomethylating agent, including azacitidine and decitabine; ribonucleotide reductase inhibitor, including hydroxyurea; and an asparagine deplete, including asparaginase.

Representative plant-derived agents include vinca alkaloids, including vincristine, vinblastine, vindesine, vinzolidine, and vinorelbine; podophyllotoxins, including etoposide and teniposide; and taxanes, including docetaxel, larotaxel, ortataxel, paclitaxel, and tesetaxel.

Representative type I topoisomerase inhibitors include camptothecins, including belotecan, irinotecan, rubitecan, and topotecan. Representative type II topoisomerase inhibitors include amsacrine, etoposide, etoposide phosphate, and teniposide, which are derivatives of epipodophyllotoxins.

Molecularly targeted therapies include biologic agents such as cytokines and other immune-regulating agents. Useful cytokines include interleukin-2 (IL-2, aldesleukin), interleukin 4 (IL-4), interleukin 12 (IL-12), and interferon, which includes more than 23 related subtypes. Other cytokines include granulocyte colony stimulating factor (CSF) (filgrastim) and granulocyte macrophage CSF (sargramostim). Other immuno-modulating agents include bacillus Calmette-Guerin, levamisole, and octreotide; monoclonal antibodies against tumor antigens, such as trastruzumab and rituximab; and cancer vaccines, which induce an immune response to tumors.

In addition, molecularly targeted drugs that interfere with specific molecules involved in tumor growth and progression include inhibitors of epidermal growth factor (EGF), transforming growth factor-alpha (TGF_{α}), TGF_{β}, heregulin, insulin-like growth factor (IGF), fibroblast growth factor (FGF), keratinocyte growth factor (KGF), colony stimulating factor (CSF), erythropoietin (EPO), interleukin-2 (IL-2), nerve growth factor (NGF), platelet-derived growth factor (PDGF), hetaptocyte growth factor (HGF), vascular endothelial growth factor (VEGF), angiopoietin, epidermal growth factor receptor (EGFR), human epidermal growth factor receptor 2 (HER2), HER4, insulin-like growth factor 1 receptor (IGF1R), IGF2R, fibroblast growth factor 1 receptor (FGF1R), FGF2R, FGF3R, FGF4R, vascular endothelial growth factor receptor (VEGFR), tyrosine kinase with immunoglobulin-like and epidermal growth factor-like domains 2 (Tie-2), platelet-derived growth factor receptor (PDGFR), Abl, Bcr-Abl, Raf, FMS-like tyrosine kinase 3 (FLT3), c-Kit, Src, protein kinase c (PKC), tropomyosin receptor kinase (Trk), Ret, mammalian target of rapamycin (mTOR), Aurora kinase, polo-like kinase (PLK), mitogen activated protein kinase (MEK), mesenchymal-epithelial transition factor (c-MET), cyclin-dependant kinase (CDK), Akt, extracellular signal-regulated kinases (ERK), poly(ADP) ribose polymerase (PARP), and the like.

Specific molecularly targeted drugs include selective estrogen receptor modulators, such as tamoxifen, toremifene, fulvestrant, and raloxifene; antiandrogens, such as bicalutamide, nilutamide, megestrol, and flutamide; and aromatase inhibitors, such as exemestane, anastrozole, and letrozole. Other specific molecularly targeted drugs include agents which inhibit signal transduction, such as imatinib, dasatinib, nilotinib, trastuzumab, gefitinib, erlotinib, cetuximab, lapatinib, panitumumab, and temsirolimus; agents that induce apoptosis, such as bortezomib; agents that block angiogenesis, such as bevacizumab, sorafenib, and sunitinib; agents that help the immune system destroy cancel cells, such as rituximab and alemtuzumab; and monoclonal antibodies which deliver toxic molecules to cancer cells, such as gemtuzumab ozogamicin, tositumomab, 131I-tositumoab, and ibritumomab tiuxetan.

Biological Activity

The activity of compounds as PHD modulators may be determined by a variety of methods, including in vitro and in vivo methods.

Inhibition of PHD2 Enzyme

The IC₅₀ values for the PHD2 enzyme (residues 181-417) were determined by mixing increasing amounts of an inhibitor with a fixed amount of enzyme (5 nM, final concentration) and Biotin-labeled peptide (Biotin-Asp-Leu-Glu-Met-Leu-Ala-Pro-Tyr-Ile-Pro-Met-Asp-Asp-Asp-Phe-Gln-Leu, 1 μM final concentration) and 2-oxyglutarate (2 μM final concentration) in 50 mM HEPES, 50 mM KCl, 0.5 mM TCEP, 2 μM FeCl₂, 0.1 mg/mL BSA, at pH 7.3. The reaction was conducted by pre-incubating the enzyme in the presence of the inhibitor for 60 minutes at room temperature. The activity of the free enzyme was measured by adding the peptide, the 2-oxoglutarate, and ascorbic acid (1 mM final concentration). The enzymatic activity was quenched after 60 minutes by adding an excess of a tight binding inhibitor to the assay mixture. The amount of product released was measured by using a LC/MS system (Agilent HPLC with Applied Biosystems API3000 Mass Spectrometer). Data were analyzed using the classical isotherm equation for the determination of IC₅₀ values and are reported in Table 1, below, as pIC₅₀, i.e., −log(IC₅₀), where IC₅₀ is molar concentration of the test compound at 50% inhibition.

Cell-based HIF-α Stabilization Assay

H9c2 rat cardiomyocytes (ATCC) were seeded in 96-well tissue culture microplates and cultured for 24 hours prior to addition of compounds (11 point range of serial dilutions) or DMSO vehicle. After 24 hours of compound incubation, whole cell extracts were prepared by lysing cells in cell extraction buffer containing protease and phosphatase inhibitors (Meso-Scale Discovery). HIF1a protein content was assessed by ELISA (Meso-Scale Discovery) and expressed as % relative to the maximum response obtained from the positive control, desferrioxamine (Sigma-Aldrich). EC₅₀ for each compound was obtained by curve-fitting using XLfit4 MicroSoft Excel curve-fitting software to calculate the compound concentration that results in 50% of the desferrioxamine maximum response. These data are reported in Table 1, below, as pEC₅₀, i.e., −log(EC₅₀), where EC₅₀ is molar concentration of the test compound at 50% desferrioxamine maximum response.

### EXAMPLES

The following examples are intended to be illustrative and non-limiting, and represent specific embodiments of the present invention.

¹H Nuclear magnetic resonance (NMR) spectra were obtained for many of the compounds in the following examples. Characteristic chemical shifts (6) are given in parts-per-million downfield from tetramethylsilane using conventional abbreviations for designation of major peaks, including s (singlet), d (doublet), t (triplet), q (quartet), m (multiplet), and br (broad). The following abbreviations are used for common solvents: CDCl₃ (deuterochloroform), DMSO-d₆ (deuterodimethylsulfoxide), CD₃OD (deuteromethanol), CD₃CN (deuteroacetonitrile), and THF-d₈ (deuterotetrahydrofuran). The mass spectra (m/z for [M+H]⁺) were recorded using either electrospray ionization (ESI-MS) or atmospheric pressure chemical ionization (APCI-MS) mass spectrometry.

Where indicated, products of certain preparations and examples are purified by mass-triggered HPLC (Pump: Waters™ 2525; MS: ZQ™; Software: MassLynx™), flash chromatography or preparative thin layer chromatography (TLC). Reverse phase chromatography is typically carried out on a column (e.g., Phenomenex Gemini™ 5μ, C18, 30 mm×150 mm; Axia™, 5μ, 30 mm×75 mm) under acidic conditions (“acid mode”) eluting with CH₃CN and water mobile phases containing 0.035% and 0.05% trifluoroacetic acid (TFA), respectively, or under basic conditions (“basic mode”) eluting with water and 20/80 (v/v) water/acetonitrile mobile phases, both containing 10 mM NH₄HCO₃. Preparative TLC is typically carried out on silica gel 60 F₂₅₄ plates. After isolation by chromatography, the solvent is removed and the product is obtained by drying in a centrifugal evaporator (e.g., GeneVac™), rotary evaporator, evacuated flask, etc. Reactions in an inert (e.g., nitrogen) or reactive (e.g., H₂) atmosphere are typically carried out at a pressure of about 1 atmosphere (14.7 psi).

### Preparation 1: 3-(benzyloxy)-5-(4-ethylpiperazine-1-carbonyl)picolinic acid

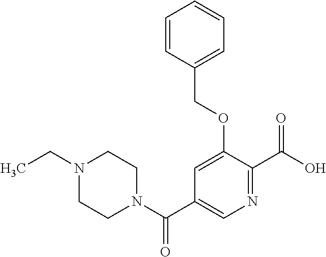 [SMILES: CCN1CCN(C(=O)c2cnc(C(=O)O)c(OCc3ccccc3)c2)CC1]

### Step A: ethyl 3-chloro-5-(4-ethylpiperazine-1-carbonyl)picolinate

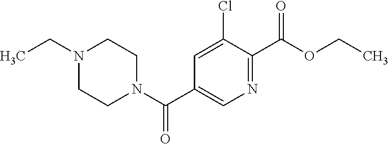 [SMILES: CCOC(=O)c1ncc(C(=O)N2CCN(CC)CC2)cc1Cl]

To a 100 mL round bottom flask equipped with a stir bar was charged 5-chloro-6-(ethoxycarbonyl)nicotinic acid (1.13 g, 4.92 mmol), EDC hydrochloride (1.226 g, 6.40 mmol), HOBt (0.980 g, 6.40 mmol), Et₃N (1.372 mL, 9.84 mmol), and DMF (16.40 mL). The reaction mixture was stirred at room temperature 5 minutes. Next 1-ethylpiperazine (0.758 mL, 5.91 mmol) was added. The reaction mixture was stirred overnight at room temperature, then diluted with ethyl acetate (30 mL) and washed with water (2×20 mL) followed by brine (20 mL). The organic layer was collected, dried over Na₂SO₄, and concentrated to a residue which was purified by silica column chromatography (120 g) eluting with a gradient of 5-40% EtOAc in heptane to give the title compound as a viscous yellow oil (1.6 g, 4.91 mmol, 100%). ¹H NMR (400 MHz, DMSO-d₆) δ ppm 1.00 (t, J=7.20 Hz, 3H), 1.33 (t, J=7.20 Hz, 3H), 2.27-2.45 (m, 6H), 3.32 (br s, 2H), 3.63 (br s, 2H), 4.40 (q, J=7.24 Hz, 2H), 8.19 (d, J=1.52 Hz, 1H), 8.56-8.67 (m, 1H); ESI-MS m/z [M+H]+ 326.1.

### Step B: 3-(benzyloxy)-5-(4-ethylpiperazine-1-carbonyl)picolinic acid

To a 20 mL vial equipped with stirring was charged ethyl 3-chloro-5-(4-ethylpiperazine-1-carbonyl)picolinate (372 mg, 1.142 mmol) and DMF (2284 μL) under nitrogen. To the stirred solution was added phenylmethanol (355 μL, 3.43 mmol) and NaH (114 mg, 2.85 mmol) at room temperature. The reaction mixture was warmed to 40° C. and stirred for 1 hour, cooled to room temperature, and quenched with water. The aqueous layer was washed with EtOAc (5 mL). The organic layer was removed, and the aqueous layer was acidified to pH 5 using 1N HCl and dried. The residue was diluted in DMSO (2 mL) and purified by preparative HPLC, giving the title compound as a tan semisolid (217 mg, 51.4%). ESI-MS m/z [M+H]⁺ 370.2.

### Preparation 2: 3-(benzyloxy)-N-(4-cyano-2-methylbenzyl)-5-(4-ethylpiperazine-1-carbonyl)picolinamide

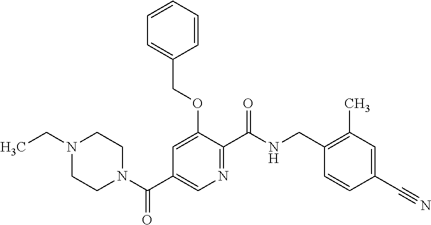 [SMILES: CCN1CCN(C(=O)c2cnc(C(=O)NCc3ccc(C#N)cc3C)c(OCc3ccccc3)c2)CC1]

To a 20 mL screw top vial equipped with a stir bar was charged 3-(benzyloxy)-5-(4-ethylpiperazine-1-carbonyl)picolinic acid (200 mg, 0.541 mmol), 1H-benzo[d][1,2,3]triazol-1-ol (95 mg, 0.704 mmol), EDC hydrochloride (135 mg, 0.704 mmol), DMF (2707 μL) and Et₃N (302 μL, 2.166 mmol). The cloudy reaction mixture was stirred 5 minutes at room temperature. Next 4-(aminomethyl)-3-methylbenzonitrile hydrochloride (198 mg, 1.083 mmol) was added. The reaction mixture was stirred at room temperature for two days, then diluted with water (2 mL) and extracted with EtOAc (2×20 mL). The organic layer was washed with brine, dried over Na₂SO₄, and concentrated to a residue, giving the title compound as a brown semisolid (105.8 mg, 39.3%). ESI-MS m/z [M+H]⁺ 498.3.

### Preparation 3: (R)-3-(benzyloxy)-N-(5-cyano-2,3-dihydro-1H-inden-1-yl)-5-(4-ethylpiperazine-1-carbonyl)picolinamide

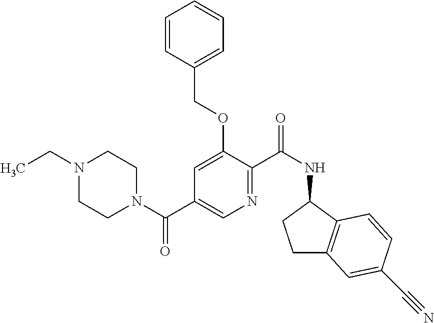 [SMILES: CCN1CCN(C(=O)c2cnc(C(=O)N[C@@H]3CCc4cc(C#N)ccc43)c(OCc3ccccc3)c2)CC1]

The title compound was prepared in a manner similar to Preparation 2, using (R)-1-amino-2,3-dihydro-1H-indene-5-carbonitrile hydrochloride in place of 4-(aminomethyl)-3-methylbenzonitrile hydrochloride. ESI-MS m/z [M+H]⁺ 510.3.

### Preparation 4: 3-chloro-5-cyano-N-(4-cyanobenzyl)picolinamide

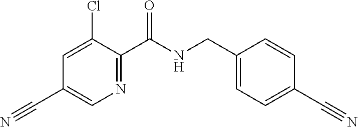 [SMILES: N#Cc1ccc(CNC(=O)c2ncc(C#N)cc2Cl)cc1]

To a 40 mL screw top vial equipped with a stir bar was added 3-chloro-5-cyanopicolinic acid (183 mg, 1 mmol), 1H-benzo[d][1,2,3]triazol-1-ol (176 mg, 1.300 mmol), EDC hydrochloride (249 mg, 1.300 mmol), DMF (5000 μL) and Et₃N (139 μL, 1.00 mmol). The cloudy reaction mixture was stirred for 5 minutes at room temperature. Next 4-(aminomethyl)benzonitrile (132 mg, 1.00 mmol) was added and the reaction mixture was stirred at room temperature overnight. Et₃N (139 μL, 1.000 mmol) was subsequently added and the reaction mixture was stirred at room temperature for 2 hours, then diluted with water (2 mL) and ethanol (2 mL) and acidified with 1 N HCl to pH 5. A precipitate was collected and dried on filter paper to give the title compound as an off-white solid (120 mg, 40.4%). ¹H NMR (400 MHz, DMSO-d₆) δ ppm 4.57 (d, J=5.81 Hz, 2H), 7.54 (d, J=8.59 Hz, 2H), 7.84 (d, J=8.59 Hz, 2H), 8.75 (d, J=1.52 Hz, 1H), 9.06 (d, J=1.52 Hz, 1H), 9.44-9.49 (m, 1H); ESI-MS m/z [M+H]⁺ 297.0.

### Preparation 5: 5-cyano-N-(4-cyanobenzyl)-3-methoxypicolinamide

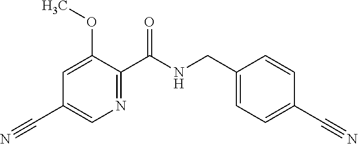 [SMILES: COc1cc(C#N)cnc1C(=O)NCc1ccc(C#N)cc1]

Sodium methoxide (634 μL, 0.317 mmol) in methanol (0.5 M) was added to a solution of 3-chloro-5-cyano-N-(4-cyanobenzyl)picolinamide (94 mg, 0.317 mmol) in acetonitrile (603 μL). The resulting solution was stirred at room temperature for 6 hours and at 60° C. for 4 hours and then filtered. The filtrate was purified by preparative HPLC, eluting with a gradient of 20-45% acetonitrile in water (containing formic acid) to give the title compound (18 mg, 19%) as an off-white solid. ¹H NMR (400 MHz, CD₃OD) δ ppm 3.97 (s, 3H), 4.59 (s, 1H), 4.65 (s, 2H), 7.56 (d, J=8.59 Hz, 2H), 7.69-7.73 (m, 2H), 8.03 (s, 1H), 8.51 (br s, 1H); ESI-MS m/z [M+H]⁺ 293.1.

### Preparation 6: tert-butyl (4-bromo-2-methylbenzyl)carbamate

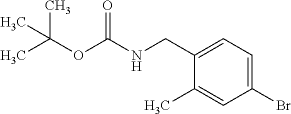 [SMILES: Cc1cc(Br)ccc1CNC(=O)OC(C)(C)C]

To a round bottom flask were added di-tert-butyl dicarbonate (11.60 mL, 50.0 mmol), NaHCO₃ (10.50 g, 125 mmol), (4-bromo-2-methylphenyl)methanamine (5 g, 24.99 mmol) and dioxane (54.9 mL). The reaction mixture was stirred at room temperature overnight, then filtered and concentrated. The residue was purified by (silica) column chromatography, eluting with a gradient of 5-30% EtOAc in heptane to give the title compound as a white solid (6.581 g, 88%). ESI-MS m/z [M+H]⁺ 300.2.

### Preparation 7: tert-butyl (4-cyano-2-methylbenzyl)carbamate

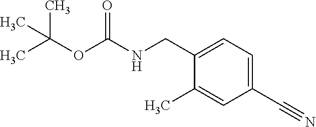 [SMILES: Cc1cc(C#N)ccc1CNC(=O)OC(C)(C)C]

To a round bottom flask equipped with stirring were added tert-butyl (4-bromo-2-methylbenzyl)carbamate (6.581 g, 21.92 mmol) and dicyanozinc (2.57 g, 21.92 mmol) followed by DMF (73.1 mL) under nitrogen. The resulting suspension was degassed for 2 minutes and then tetrakis(triphenylphosphine)palladium(0) (1.267 g, 1.096 mmol) was added. The reaction mixture was degassed for 2 more minutes, then heated to 100° C. and stirred overnight. The reaction mixture was subsequently cooled to room temperature and poured into a separatory funnel containing water (76 mL). Ethyl acetate (113 mL) was added. After shaking, the resulting emulsion was filtered through paper to help separate the layers. The organic layer was collected, washed with brine, dried over Na₂SO₄ and concentrated to an oil. The oil was purified using a Moritex™ column (240 g silica) eluting with a gradient of 5-30% EtOAc in heptane to give the title compound as a white solid (3.972 g, 73.6%). ESI-MS m/z [M+H]⁺ 247.1.

### Preparation 8: 4-(aminomethyl)-3-methylbenzonitrile

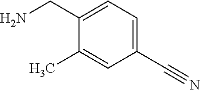

To a round bottom flask containing tert-butyl (4-cyano-2-methylbenzyl)carbamate (3.972 g, 16.13 mmol) was added HCl in dioxane (57.6 mL, 230 mmol). The reaction mixture was stirred for 2 hours at room temperature. UPLC indicated conversion to the desired product. The crude reaction was dried under vacuum to give an HCL salt of the title compound as an off-white solid (3.109 g). ¹H NMR (400 MHz, DMSO-d₆) δ ppm 2.38 (s, 3H), 4.11 (br s, 2H), 7.55 (d, J=7.83 Hz, 1H), 7.74-7.81 (m, 2H), 8.28 (br s, 2H); ESI-MS m/z [M+H]⁺ 147.0.

### Preparation 9: 3-chloro-5-cyano-N-(4-cyano-2-methylbenzyl)picolinamide

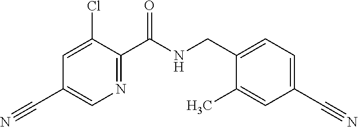 [SMILES: Cc1cc(C#N)ccc1CNC(=O)c1ncc(C#N)cc1Cl]

To a 40 mL screw top vial equipped with stir bar were added 3-chloro-5-cyanopicolinic acid (548 mg, 3 mmol), 1H-benzo[d][1,2,3]triazol-1-ol (527 mg, 3.90 mmol), EDC hydrochloride (748 mg, 3.90 mmol), DMF (15 mL) and Et₃N (836 μL, 6.00 mmol). The cloudy reaction mixture was stirred 5 for minutes at room temperature. Next 4-(aminomethyl)-3-methylbenzonitrile hydrochloride (548 mg, 3.00 mmol) was added. The reaction mixture was stirred at room temperature for 72 hours, then diluted with water (6 mL) and ethanol (6 mL) and acidified with 1N HCl to pH 5. A precipitate was collected and dried on the filter paper to give the title compound as a white solid (386 mg, 41.4%). ¹H NMR (400 MHz, DMSO-d₆) δ ppm 2.38 (s, 3H), 4.53 (d, J=6.06 Hz, 2H), 7.46-7.50 (m, 1H), 7.67-7.71 (m, 2H), 8.75 (d, J=1.77 Hz, 1H), 9.07 (d, J=1.77 Hz, 1H), 9.37 (t, J=5.94 Hz, 1H); ESI-MS m/z [M+H]⁺ 311.2.

### Preparation 10: 5-cyano-N-(4-cyano-2-methylbenzyl)-3-methoxypicolinamide

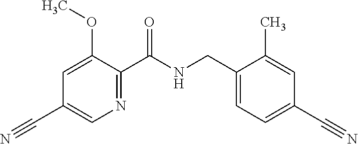 [SMILES: COc1cc(C#N)cnc1C(=O)NCc1ccc(C#N)cc1C]

To 3-chloro-5-cyano-N-(4-cyano-2-methylbenzyl)picolinamide (379 mg, 1.220 mmol) in acetonitrile (2323 μL) was added a 0.5 M solution of sodium methoxide (2439 μL, 1.220 mmol) in methanol. The resulting solution was stirred at 50° C. overnight and then filtered. The filtrate was purified by preparative HPLC, eluting with a gradient of 20-45% acetonitrile in water (containing TFA) to give the title compound as an off-white solid (144 mg, 38.5%). ¹H NMR (400 MHz, DMSO-d₆) δ ppm 2.36 (s, 3H), 3.91 (s, 3H), 4.48 (d, J=5.81 Hz, 2H), 7.48 (d, J=7.83 Hz, 1H), 7.65-7.72 (m, 2H), 8.17 (d, J=1.52 Hz, 1H), 8.63 (d, J=1.52 Hz, 1H), 9.11 (t, J=6.06 Hz, 1H); ESI-MS m/z (M+H)⁺307.3.

### Preparation 11: 6-(bromomethyl)nicotinonitrile

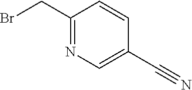

A slurry of 6-methylnicotinonitrile (500 mg, 4.23 mmol), N-bromosuccinimide (791 mg, 4.44 mmol) and AIBN (208 mg, 1.270 mmol) in CCl₄ (5 mL) was heated at 85° C. for 20 hours. The mixture was concentrated in vacuo and the residue was purified by flash column chromatography on silica gel (40 g SiO₂) eluting with a gradient of 0-40% EtOAc in hexanes to give the title compound as a light red oil (441 mg, 52.9%). ¹H NMR (400 MHz, CDCl₃) δ ppm 4.58 (s, 2H), 7.60 (dd, J=8.1, 0.8 Hz, 1H), 7.99 (dd, J=8.1, 2.0 Hz, 1H), 8.82-8.88 (m, 1H); ESI-MS m/z [M+H]⁺ 197, 199.

### Preparation 12: 6-(N,N-(bis-tert-butoxycarbonyl)aminomethyl)nicotinonitrile

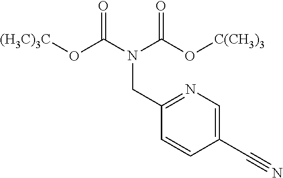 [SMILES: CC(C)(C)OC(=O)N(Cc1ccc(C#N)cn1)C(=O)OC(C)(C)C]

A solution of N,N-(bis-tert-butoxycarbonyl)amine (582 mg, 2.68 mmol) in THF (8 mL) was added to NaH (125 mg, 3.13 mmol) at 0° C. Next 6-(bromomethyl)nicotinonitrile (440 mg, 2.233 mmol) in THF (8 mL) at 0° C. was added, and the solution was allowed to warm to 25° C. The reaction mixture was stirred at this temperature for 16 hours and then concentrated in vacuo. The residue was taken up in EtOAc (100 mL), washed with saturated (aq) ammonium chloride (100 mL) and brine, dried over MgSO₂, and concentrated in vacuo. The crude material was purified by flash column chromatography on silica gel (40 g SiO₂) eluting with a gradient of 0-40% EtOAc in hexanes to give the title compound as a light yellow solid (415 mg, 55.7%). ¹H NMR (400 MHz, CDCl₃) δ ppm 1.47 (s, 18H), 4.99 (s, 2H), 7.32 (dd, J=8.2, 0.6 Hz, 1H), 7.93 (dd, J=8.2, 2.1 Hz, 1H), 8.82 (dd, J=2.0, 0.8 Hz, 1H); ESI-MS m/z [M+H]⁺ 334.

### Preparation 13: 6-(aminomethyl)nicotinonitrile

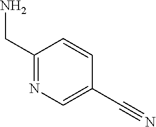

To a solution of 6-(N,N-(bis-tert-butoxycarbonyl)aminomethyl)nicotinonitrile (410 mg, 1.230 mmol) in DCM (8 mL) was added 4 M HCl in dioxane (1.0 mL, 4.00 mmol). The solution was stirred at 20° C. for 19 hours, after which an additional portion 4 M HCl in dioxane (0.5 mL) was added. The reaction mixture was stirred for 3 days and then concentrated in vacuo to give an HCl salt of the title compound as a yellow solid which was used without further purification (215 mg). ¹H NMR (400 MHz, DMSO-d₆) δ ppm 4.33 (q, J=5.8 Hz, 2H), 7.74 (d, J=8.1 Hz, 1H), 8.42 (dd, J=8.2, 2.1 Hz, 1H), 8.57 (br s, 3H), 9.12 (dd, J=2.0, 0.8 Hz, 1H); ESI-MS m/z [M+H]⁺ 134.

### Preparation 14: 3-bromo-5-chloro-N-(4-cyano-2,6-dimethylbenzyl)picolinamide

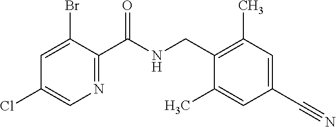 [SMILES: Cc1cc(C#N)cc(C)c1CNC(=O)c1ncc(Cl)cc1Br]

To a screw top vial equipped with a stir bar was added 3-bromo-5-chloropicolinic acid (236 mg, 1 mmol), 1H-benzo[d][1,2,3]triazol-1-ol (176 mg, 1.300 mmol), EDC hydrochloride (249 mg, 1.300 mmol), DMF (5 mL) and Et₃N (139 μL, 1.00 mmol). The reaction mixture was stirred for 5 minutes at room temperature. Next 4-(aminomethyl)-3,5-dimethylbenzonitrile (160 mg, 1.000 mmol) was added. The reaction mixture was stirred at room temperature overnight, then heated at 60° C. for 1 hour, and filtered. The filtrate was purified by preparative HPLC, eluting with a gradient of 40-65% acetonitrile in water (containing TFA) to give the title compound as an off-white solid (171 mg, 45.2%). ESI-MS m/z [M+H]⁺ 378.0.

### Preparation 15: 5-chloro-N-(4-cyano-2,6-dimethylbenzyl)-3-methoxypicolinamide

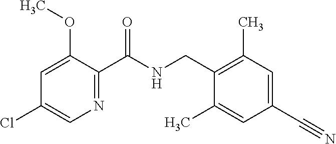 [SMILES: COc1cc(Cl)cnc1C(=O)NCc1c(C)cc(C#N)cc1C]

To a mixture of 3-bromo-5-chloro-N-(4-cyano-2,6-dimethylbenzyl)picolinamide (133 mg, 0.351 mmol) in methanol (1405 μL) was added a 0.5 M solution of sodium methoxide (702 μL, 0.351 mmol) in methanol. The reaction mixture was stirred at 50° C. overnight. On two successive days, additional sodium methoxide (702 μL, 0.351 mmol) in methanol (0.5 M) was added. After each addition the reaction mixture was stirred at 50° C. overnight. Following the last overnight reaction, the reaction mixture was combined with a separate preparation and filtered. The filtrate was purified by preparative HPLC, eluting with a gradient of 35-60% acetonitrile in water (containing TFA) to give the title compound as an off-white solid (77 mg, 67%). ESI-MS m/z [M+H]⁺ 330.1.

### Preparation 16: 3-chloro-5-cyano-N-(4-cyano-2,6-dimethylbenzyl)picolinamide

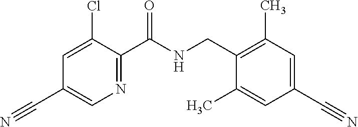 [SMILES: Cc1cc(C#N)cc(C)c1CNC(=O)c1ncc(C#N)cc1Cl]

To a screw top vial equipped with a stir bar was added 3-chloro-5-cyanopicolinic acid (183 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred 5 minutes at room temperature. Next 4-(aminomethyl)-3,5-dimethylbenzonitrile (160 mg, 1.00 mmol) was added. The reaction was stirred at room temperature for 72 hours and then filtered. The filtrate was purified by preparative HPLC, eluting with a gradient of 40-65% acetonitrile in water (containing formic acid) to give the title compound as an off-white solid (75 mg, 23%). ESI-MS m/z [M+H]⁺ 325.0.

### Preparation 17: 5-cyano-N-(4-cyano-2,6-dimethylbenzyl)-3-methoxypicolinamide

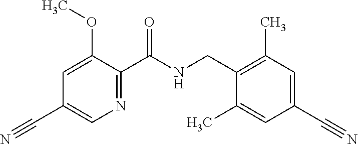 [SMILES: COc1cc(C#N)cnc1C(=O)NCc1c(C)cc(C#N)cc1C]

To a mixture of 3-chloro-5-cyano-N-(4-cyano-2,6-dimethylbenzyl)picolinamide (75 mg, 0.231 mmol) in methanol (924 μL) was added a 0.5 M solution of sodium methoxide (462 μL, 0.231 mmol) in methanol. The reaction mixture was stirred at 50° C. overnight. On two successive days, additional sodium methoxide (462 μL, 0.231 mmol) in methanol (0.5 M) was added. After each addition the reaction mixture was stirred at 50° C. overnight. Following the third overnight reaction, the reaction mixture was filtered. The filtrate was purified by preparative HPLC, eluting with a gradient of 20-45% acetonitrile in water (containing TFA) to give the title compound as an off-white solid (12 mg, 16%). ESI-MS m/z [M+H]⁺ 321.2.

### Preparation 18: N-(4-cyano-2,6-dimethylbenzyl)-3,5-difluoropicolinamide

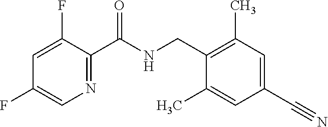 [SMILES: Cc1cc(C#N)cc(C)c1CNC(=O)c1ncc(F)cc1F]

To a screw top vial equipped with a stir bar was added 3,5-difluoropicolinic acid (159 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (139 μL, 1.00 mmol). The reaction mixture was stirred for 5 minutes at room temperature. Next 4-(aminomethyl)-3,5-dimethylbenzonitrile (160 mg, 1.00 mmol) was added. The reaction mixture was stirred at room temperature overnight and then filtered. The filtrate was purified by preparative HPLC, eluting with a gradient of 35-60% acetonitrile in water (containing TFA) to the title compound as an off-white solid (196 mg, 65.1%). ESI-MS m/z [M+H]⁺ 302.2.

### Preparation 19: N-(4-cyano-2,6-dimethylbenzyl)-5-fluoro-3-methoxypicolinamide and N-(4-cyano-2,6-dimethylbenzyl)-3-fluoro-5-methoxypicolinamide

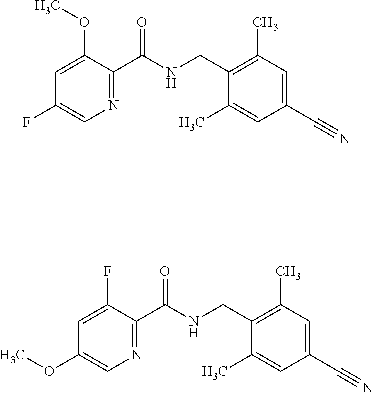 [SMILES: COc1cc(F)cnc1C(=O)NCc1c(C)cc(C#N)cc1C ; COc1cnc(C(=O)NCc2c(C)cc(C#N)cc2C)c(F)c1]

To a mixture of N-(4-cyano-2,6-dimethylbenzyl)-3,5-difluoropicolinamide (196 mg, 0.651 mmol) in acetonitrile (1239 μL) was added a 0.5 M solution of sodium methoxide (1301 μL, 0.651 mmol) in methanol. The reaction mixture was stirred at room temperature for 2 hours and then filtered. The filtrate was purified by preparative HPLC, eluting with a gradient of 25-50% acetonitrile in water (containing TFA) to give N-(4-cyano-2,6-dimethylbenzyl)-5-fluoro-3-methoxypicolinamide as an off-white solid (94 mg, 46%); ESI-MS m/z [M+H]⁺ 314.2; and N-(4-cyano-2,6-dimethylbenzyl)-3-fluoro-5-methoxypicolinamide as an off-white solid (49 mg, 24%); ESI-MS m/z [M+H]⁺ 314.2.

### Preparation 20: 3-chloro-N-(4-cyano-2,6-dimethylbenzyl)-5-(trifluoromethyl)picolinamide

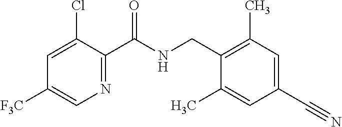 [SMILES: Cc1cc(C#N)cc(C)c1CNC(=O)c1ncc(C(F)(F)F)cc1Cl]

To a screw top vial equipped with a stir bar was added 3-chloro-5-(trifluoromethyl)picolinic acid (226 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3,5-dimethylbenzonitrile hydrochloride (256 mg, 1.300 mmol) was added. The reaction mixture was stirred at room temperature overnight and then filtered. The filtrate was purified by preparative HPLC, eluting with a gradient of 40-65% acetonitrile in water (containing TFA) to give the title compound as an off-white solid (81 mg, 22%). ESI-MS m/z [M+H]⁺ 368.1.

### Preparation 21: N-(4-cyano-2,6-dimethylbenzyl)-3-methoxy-5-(trifluoromethyl)picolinamide

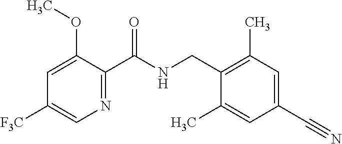 [SMILES: COc1cc(C(F)(F)F)cnc1C(=O)NCc1c(C)cc(C#N)cc1C]

To a mixture of 3-chloro-N-(4-cyano-2,6-dimethylbenzyl)-5-(trifluoromethyl)picolinamide (81 mg, 0.220 mmol) in acetonitrile (420 μL) was added a 0.5 M solution of sodium methoxide (1322 μL, 0.661 mmol) in methanol. The reaction mixture was stirred at 50° C. overnight and then filtered. The filtrate was purified by preparative HPLC, eluting with a gradient of 35-60% acetonitrile in water (containing TFA) to give the title compound as an off-white solid (54 mg, 68%). ESI-MS m/z [M+H]⁺ 364.2.

### Preparation 22: N-(4-cyano-2,6-dimethylbenzyl)-3-fluoro-5-methoxypicolinamide

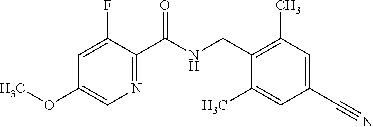 [SMILES: COc1cnc(C(=O)NCc2c(C)cc(C#N)cc2C)c(F)c1]

To a screw top vial equipped with a stir bar was added 3-fluoro-5-methoxypicolinic acid (171 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3,5-dimethylbenzonitrile hydrochloride (256 mg, 1.30 mmol) was added. The reaction mixture was stirred at room temperature overnight and then filtered. The filtrate was purified by preparative HPLC, eluting with a gradient of 35-60% acetonitrile in water (containing TFA) to give the title compound as an off-white solid (114 mg, 36.4%). ESI-MS m/z [M+H]⁺314.2.

### Preparation 23: N-(4-cyano-2,6-dimethylbenzyl)-3,5-dimethoxypicolinamide

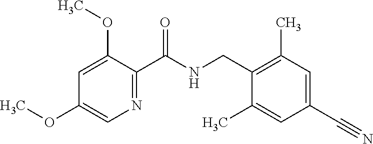 [SMILES: COc1cnc(C(=O)NCc2c(C)cc(C#N)cc2C)c(OC)c1]

To a mixture of N-(4-cyano-2,6-dimethylbenzyl)-3-fluoro-5-methoxypicolinamide (114 mg, 0.364 mmol) in acetonitrile (693 μL) was added a 0.5 M solution of sodium methoxide (2183 μL, 1.092 mmol) in methanol. The reaction mixture was stirred at 50° C. overnight and then filtered. The filtrate was purified by preparative HPLC, eluting with a gradient of 25-50% acetonitrile in water (containing TFA) to give the title compound as an off-white solid (104 mg, 88%). ESI-MS m/z [M+H]⁺ 326.2.

### Preparation 24: (R)—N-(5-cyano-2,3-dihydro-1H-inden-1-yl)-3,5-difluoropicolinamide

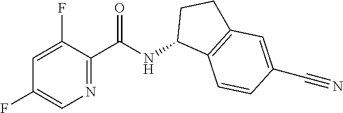 [SMILES: N#Cc1ccc2c(c1)CC[C@H]2NC(=O)c1ncc(F)cc1F]

To a screw top vial equipped with a stir bar was added 3,5-difluoropicolinic acid (159 mg, 1 mmol), 1H-benzo[d][1,2,3]triazol-1-ol (176 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next (R)-1-amino-2,3-dihydro-1H-indene-5-carbonitrile hydrochloride (253 mg, 1.300 mmol) was added. The reaction mixture was stirred at room temperature overnight and then filtered. The filtrate was purified by preparative HPLC, eluting with a gradient of 25-50% acetonitrile in water (containing TFA) to give the title compound as an off-white solid (97 mg, 32%). ESI-MS m/z [M+H]⁺ 300.0.

### Preparation 25: (R)—N-(5-cyano-2,3-dihydro-1H-inden-1-yl)-5-fluoro-3-methoxypicolinamide

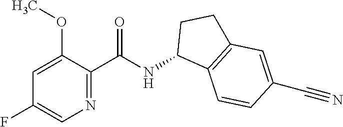 [SMILES: COc1cc(F)cnc1C(=O)N[C@@H]1CCc2cc(C#N)ccc21]

To a mixture of (R)—N-(5-cyano-2,3-dihydro-1H-inden-1-yl)-3,5-difluoropicolinamide (97 mg, 0.324 mmol) in acetonitrile (617 μL) was added a 0.5 M solution of sodium methoxide (648 μL, 0.324 mmol) in methanol. The reaction mixture was stirred at room temperature overnight and then filtered. The filtrate was purified by preparative HPLC, eluting with a gradient of 25-50% acetonitrile in water (containing TFA) to give the title compound as an off-white solid (51 mg, 51%). ESI-MS m/z [M+H]+ 312.2.

### Preparation 26: (R)-3-bromo-5-chloro-N-(5-cyano-2,3-dihydro-1H-inden-1-yl)picolinamide

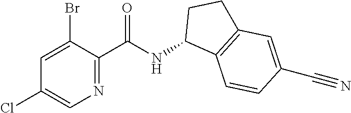 [SMILES: N#Cc1ccc2c(c1)CC[C@H]2NC(=O)c1ncc(Cl)cc1Br]

To a screw top vial equipped with a stir bar was added 3-bromo-5-chloropicolinic acid (236 mg, 1 mmol), 1H-benzo[d][1,2,3]triazol-1-ol (176 mg, 1.30 mmol), EDC hydrochloride (249 mg, 1.30 mmol), DMF (5 mL) and Et₃N (418 μL, 3.00 mmol). The reaction mixture was stirred for 5 minutes at room temperature. Next (R)-1-amino-2,3-dihydro-1H-indene-5-carbonitrile hydrochloride (253 mg, 1.300 mmol) was added. The reaction mixture was stirred at room temperature overnight and then filtered. The filtrate was purified by preparative HPLC, eluting with a gradient of 35-60% acetonitrile in water (containing TFA) to give the title compound as an off-white solid (73 mg, 19%). ESI-MS m/z [M+H]⁺ 376.1.

### Preparation 27: (R)-5-chloro-N-(5-cyano-2,3-dihydro-1H-inden-1-yl)-3-methoxypicolinamide

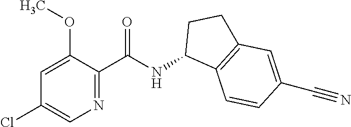 [SMILES: COc1cc(Cl)cnc1C(=O)N[C@@H]1CCc2cc(C#N)ccc21]

To a mixture of (R)-3-bromo-5-chloro-N-(5-cyano-2,3-dihydro-1H-inden-1-yl)picolinamide (73 mg, 0.194 mmol) in acetonitrile (369 μL) was added a 0.5 M solution of sodium methoxide (1163 μL, 0.581 mmol) in methanol. The reaction mixture was stirred at 50° C. overnight and then filtered. The filtrate was purified by preparative HPLC, eluting with a gradient of 25-50% acetonitrile in water (containing TFA) to give the title compound as an off-white solid (34 mg, 54%). ESI-MS m/z [M+H]⁺ 328.1.

### Preparation 28: (R)-3-chloro-N-(5-cyano-2,3-dihydro-1H-inden-1-yl)-5-(trifluoromethyl)picolinamide

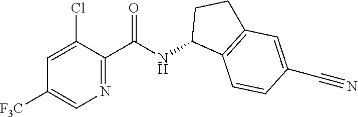 [SMILES: N#Cc1ccc2c(c1)CC[C@H]2NC(=O)c1ncc(C(F)(F)F)cc1Cl]

To a screw top vial equipped with a stir bar was added 3-chloro-5-(trifluoromethyl)picolinic acid (226 mg, 1 mmol), 1H-benzo[d][1,2,3]triazol-1-ol (176 mg, 1.30 mmol), EDC hydrochloride (249 mg, 1.30 mmol), DMF (5 mL) and Et₃N (418 μL, 3.00 mmol). The reaction mixture was stirred for 5 minutes at room temperature. Next (R)-1-amino-2,3-dihydro-1H-indene-5-carbonitrile hydrochloride (253 mg, 1.30 mmol) was added. The reaction mixture was stirred at room temperature overnight and then filtered. The filtrate was purified by preparative HPLC, eluting with a gradient of 35-60% acetonitrile in water (containing TFA) to give the title compound as an off-white solid (102 mg, 27.9%). ESI-MS m/z [M+H]⁺ 366.0.

### Preparation 29: (R)—N-(5-cyano-2,3-dihydro-1H-inden-1-yl)-3-methoxy-5-(trifluoromethyl)picolinamide

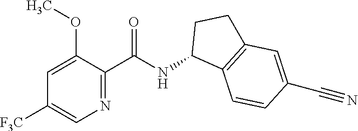 [SMILES: COc1cc(C(F)(F)F)cnc1C(=O)N[C@@H]1CCc2cc(C#N)ccc21]

To a mixture of (R)-3-chloro-N-(5-cyano-2,3-dihydro-1H-inden-1-yl)-5-(trifluoromethyl)picolinamide (102 mg, 0.279 mmol) in acetonitrile (531 μL) was added a 0.5 solution of sodium methoxide (1673 μL, 0.837 mmol) in methanol. The reaction mixture was stirred at 50° C. overnight and then filtered. The filtrate was purified by preparative HPLC, eluting with a gradient of 35-6₀% acetonitrile in water (containing TFA) to give the title compound as an off-white solid (68 mg, 68%). ESI-MS m/z [M+H]⁺ 362.2.

### Preparation 30: (R)—N-(5-cyano-2,3-dihydro-1H-inden-1-yl)-3-fluoro-5-methoxypicolinamide

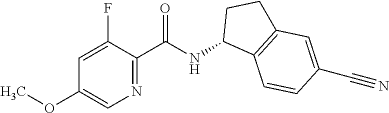 [SMILES: COc1cnc(C(=O)N[C@@H]2CCc3cc(C#N)ccc32)c(F)c1]

To a screw top vial equipped with a stir bar was added 3-fluoro-5-methoxypicolinic acid (171 mg, 1 mmol), 1H-benzo[d][1,2,3]triazol-1-ol (176 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next (R)-1-amino-2,3-dihydro-1H-indene-5-carbonitrile hydrochloride (253 mg, 1.300 mmol) was added. The reaction mixture was stirred at room temperature overnight and then filtered. The filtrate was purified by preparative HPLC, eluting with a gradient of 35-60% acetonitrile in water (containing TFA) to give the title compound as an off-white solid (152 mg, 48.8%). ESI-MS m/z [M+H]⁺ 312.2.

### Preparation 31: (R)—N-(5-cyano-2,3-dihydro-1H-inden-1-yl)-3,5-dimethoxypicolinamide

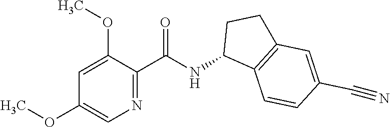 [SMILES: COc1cnc(C(=O)N[C@@H]2CCc3cc(C#N)ccc32)c(OC)c1]

To a mixture of (R)—N-(5-cyano-2,3-dihydro-1H-inden-1-yl)-3-fluoro-5-methoxypicolinamide (152 mg, 0.488 mmol) in acetonitrile (930 μL) was added a 0.5 M solution of sodium methoxide (2930 μL, 1.465 mmol) in methanol. The reaction mixture was stirred at 50° C. overnight and then filtered. The filtrate was purified by preparative HPLC, eluting with a gradient of 20-45% acetonitrile in water (containing TFA) to give the title compound as an off-white solid (97 mg, 61%). ESI-MS m/z [M+H]⁺ 324.2.

### Preparation 32: (R)-3-chloro-5-cyano-N-(5-cyano-2,3-dihydro-1H-inden-1-yl)picolinamide

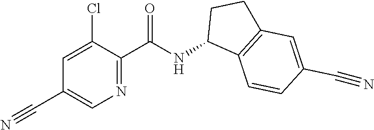 [SMILES: N#Cc1cnc(C(=O)N[C@@H]2CCc3cc(C#N)ccc32)c(Cl)c1]

To a screw top vial equipped with a stir bar was added 3-chloro-5-cyanopicolinic acid (183 mg, 1 mmol), 1H-benzo[d][1,2,3]triazol-1-ol (176 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next (R)-1-amino-2,3-dihydro-1H-indene-5-carbonitrile hydrochloride (253 mg, 1.300 mmol) was added. The reaction mixture was stirred at room temperature overnight and then filtered. The filtrate was purified by preparative HPLC, eluting with a gradient of 25-50% acetonitrile in water (containing TFA) to give the title compound as an off-white solid (68 mg, 21%). ESI-MS m/z [M+H]⁺ 323.0.

### Preparation 33: (R)-5-cyano-N-(5-cyano-2,3-dihydro-1H-inden-1-yl)-3-methoxypicolinamide

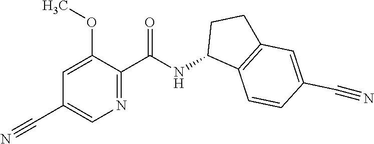 [SMILES: COc1cc(C#N)cnc1C(=O)N[C@@H]1CCc2cc(C#N)ccc21]

To a mixture of (R)-3-chloro-5-cyano-N-(5-cyano-2,3-dihydro-1H-inden-1-yl)picolinamide (68 mg, 0.211 mmol) in acetonitrile (401 μL) was added a 0.5 M solution of sodium methoxide (1264 μL, 0.632 mmol) in methanol. The reaction mixture was stirred at 50° C. overnight and then filtered. The filtrate was purified by preparative HPLC, eluting with a gradient of 25-50% acetonitrile in water (containing TFA) to give the title compound as an off-white solid (8 mg, 12%). ESI-MS m/z [M+H]⁺ 319.2.

### Preparation 34: N-(4-cyano-2-methylbenzyl)-3,5-difluoropicolinamide

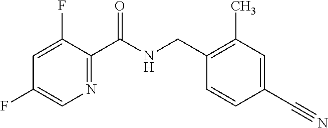 [SMILES: Cc1cc(C#N)ccc1CNC(=O)c1ncc(F)cc1F]

To a screw top vial equipped with a stir bar was added 3,5-difluoropicolinic acid (159 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3-methylbenzonitrile hydrochloride (237 mg, 1.300 mmol) was added. The reaction mixture was stirred at room temperature overnight and then filtered. The filtrate was purified by preparative HPLC, eluting with a gradient of 25-50% acetonitrile in water (containing formic acid) to give the title compound as an off-white solid (76 mg, 027%). ESI-MS m/z [M+H]⁺288.1.

### Preparation 35: N-(4-cyano-2-methylbenzyl)-5-fluoro-3-methoxypicolinamide

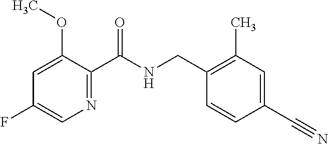 [SMILES: COc1cc(F)cnc1C(=O)NCc1ccc(C#N)cc1C]

To a mixture of N-(4-cyano-2-methylbenzyl)-3,5-difluoropicolinamide (76 mg, 0.265 mmol) in acetonitrile (504 μL) was added a 0.5 M solution of sodium methoxide (529 μL, 0.265 mmol) in methanol. The reaction mixture was stirred at room temperature overnight and then filtered. The filtrate was purified by preparative HPLC, eluting with a gradient of 25-50% acetonitrile in water (containing TFA) to give the title compound as an off-white solid (20 mg, 25%). ESI-MS m/z [M+H]+ 300.2.

### Preparation 36: 3-bromo-5-chloro-N-(4-cyano-2-methylbenzyl)picolinamide

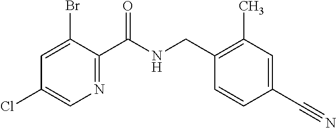 [SMILES: Cc1cc(C#N)ccc1CNC(=O)c1ncc(Cl)cc1Br]

To a screw top vial equipped with a stir bar was added 3-bromo-5-chloropicolinic acid (236 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3-methylbenzonitrile hydrochloride (237 mg, 1.300 mmol) was added. The reaction mixture was stirred at room temperature overnight and then filtered. The filtrate was purified by preparative HPLC, eluting with a gradient of 40-65% acetonitrile in water (containing formic acid) to give the title compound as an off-white solid (86 mg, 24%). ESI-MS m/z [M+H]⁺364.0.

### Preparation 37: 5-chloro-N-(4-cyano-2-methylbenzyl)-3-methoxypicolinamide

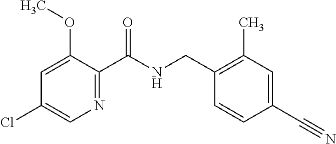 [SMILES: COc1cc(Cl)cnc1C(=O)NCc1ccc(C#N)cc1C]

To a mixture of 3-bromo-5-chloro-N-(4-cyano-2-methylbenzyl)picolinamide (86 mg, 0.236 mmol) in acetonitrile (449 μL) was added a 0.5 M solution of sodium methoxide (1415 μL, 0.708 mmol) in methanol. The reaction mixture was stirred at room temperature for 2 hours and then filtered. The filtrate was purified by preparative HPLC, eluting with a gradient of 25-50% acetonitrile in water (containing TFA) to give the title compound as an off-white solid (44 mg, 59%). ESI-MS m/z [M+H]⁺ 316.1.

### Preparation 38: 3-chloro-N-(4-cyano-2-methylbenzyl)-5-(trifluoromethyl)picolinamide

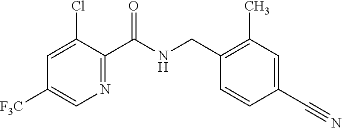 [SMILES: Cc1cc(C#N)ccc1CNC(=O)c1ncc(C(F)(F)F)cc1Cl]

To a screw top vial equipped with a stir bar was added 3-chloro-5-(trifluoromethyl)picolinic acid (226 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3-methylbenzonitrile hydrochloride (237 mg, 1.300 mmol) was added. The reaction mixture was stirred at room temperature overnight and then filtered. The filtrate was purified by preparative HPLC, eluting with a gradient of 40-65% acetonitrile in water (containing formic acid) to give the title compound as an off-white solid (72 mg, 20%). ESI-MS m/z [M+H]⁺ 354.1.

### Preparation 39: N-(4-cyano-2-methylbenzyl)-3-methoxy-5-(trifluoromethyl)picolinamide

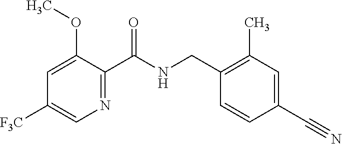 [SMILES: COc1cc(C(F)(F)F)cnc1C(=O)NCc1ccc(C#N)cc1C]

To a mixture of 3-chloro-N-(4-cyano-2-methylbenzyl)-5-(trifluoromethyl)picolinamide (72 mg, 0.204 mmol) in acetonitrile (388 μL) was added a 0.5 M solution of sodium methoxide (1221 μL, 0.611 mmol) in methanol. The reaction mixture was stirred at 50° C. overnight and then filtered. The filtrate was purified by preparative HPLC, eluting with a gradient of 25-50% acetonitrile in water (containing TFA) to give the title compound as an off-white solid (51 mg, 72%). ESI-MS m/z [M+H]⁺ 350.2.

### Preparation 40: N-(4-cyano-2-methylbenzyl)-3-fluoro-5-methoxypicolinamide

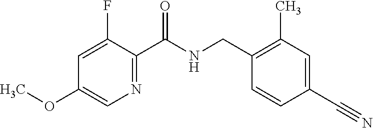 [SMILES: COc1cnc(C(=O)NCc2ccc(C#N)cc2C)c(F)c1]

To a screw top vial equipped with a stir bar was added 3-fluoro-5-methoxypicolinic acid (171 mg, 1 mmol), 1H-benzo[d][1,2,3]triazol-1-ol (204 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 4-(aminomethyl)-3-methylbenzonitrile hydrochloride (237 mg, 1.300 mmol) was added. The reaction mixture was stirred at room temperature overnight and then filtered. The filtrate was purified by preparative HPLC, eluting with a gradient of 25-50% acetonitrile in water (containing formic acid) to give the title compound as an off-white solid (136 mg, 45.4%). ESI-MS m/z [M+H]⁺300.2.

### Preparation 41: N-(4-cyano-2-methylbenzyl)-3,5-dimethoxypicolinamide

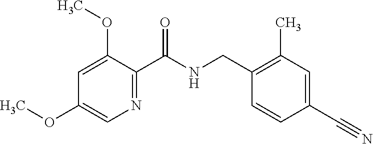 [SMILES: COc1cnc(C(=O)NCc2ccc(C#N)cc2C)c(OC)c1]

To a mixture of N-(4-cyano-2-methylbenzyl)-3-fluoro-5-methoxypicolinamide (136 mg, 0.454 mmol) in acetonitrile (866 μL) was added a 0.5 M solution of sodium methoxide (2726 μL, 1.363 mmol) in methanol. The reaction mixture was stirred at 50° C. overnight and then filtered. The filtrate was purified by preparative HPLC, eluting with a gradient of 35-60% acetonitrile in water (containing formic acid) to give the title compound as an off-white solid (67 mg, 47%). ESI-MS m/z [M+H]⁺ 312.2.

### Preparation 42: 3-(benzyloxy)-5-bromo-N-(4-cyano-2,6-dimethylbenzyl)picolinamide

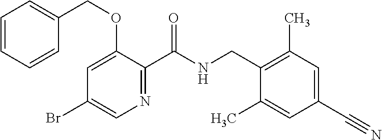 [SMILES: Cc1cc(C#N)cc(C)c1CNC(=O)c1ncc(Br)cc1OCc1ccccc1]

### Step A: 3-(benzyloxy)-5-bromopicolinonitrile

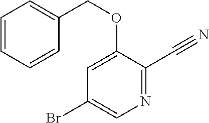 [SMILES: N#Cc1ncc(Br)cc1OCc1ccccc1]

A 60% dispersion of sodium hydride in mineral oil (3.40 g, 85 mmol) was slurried in THF (100 mL) and cooled to 0° C. To the slurry was added phenylmethanol (8.44 mL, 81 mmol) at a rate which ensured the internal temperature did not exceed 5° C. Upon completion of the NaH addition the temperature was raised to 20° C. and the reaction mixture was stirred until no visible off-gassing was observed. The reaction mixture was slowly added to a solution of 5-bromo-3-nitropicolinonitrile (16.15 g, 70.8 mmol) in THF (125 mL) while keeping the internal temperature below 30° C. Upon completion of the nitrile addition, the reaction mixture was allowed to stir at 20° C. for 30 minutes and was subsequently partitioned between water (300 mL) and isopropyl acetate (300 mL). The organic layer was separated and the aqueous phase was extracted with isopropyl acetate (2×150 mL). The organic layers were combined, washed with saturated NaCl (aq) (250 mL), dried over Na₂SO₄, filtered, and concentrated in vacuo to give a red solid. The red solid was dissolved in isopropyl acetate (110 mL) at 80° C. and cooled to 52° C. Heptane (51 mL) was added and the solution was stirred at 52° C. for 1 hour and then heated to 90° C. A suspension formed which was cooled to 10° C. The solid was filtered, washed with 50% heptane in IPAc, and dried under vacuum to give the title compound (13.5 g, 66%). ¹H NMR (400 MHz, CDCl₃) δ ppm 8.37 (d, J=1.77 Hz, 1H), 7.57 (d, J=1.77 Hz, 1H), 7.37-7.49 (m, 5H), 5.26 (s, 2H); ESI-MS m/z [M+H]⁺289.1 (⁷⁹Br).

### Step B: 3-(benzyloxy)-5-bromopicolinic acid

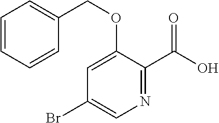 [SMILES: O=C(O)c1ncc(Br)cc1OCc1ccccc1]

A suspension of 3-(benzyloxy)-5-bromopicolinonitrile (13.4 g, 46.3 mmol), ethanol (75 mL), water (50 mL) and 50% w/w NaOH (aq) (23.32 mL, 440 mmol) was stirred for 4 hours at reflux (84° C. bath) and then allowed to cool. Ethanol was removed under reduced pressure. The resulting suspension was diluted with water (150 mL) and acidified with 6M HCl (aq) until all of the solids were dissolved. The solution was washed with isopropyl acetate (1×100 mL) and further acidified with 6M HCl (aq) to about pH 4 whereupon a solid formed. The mixture was stirred vigorously, resulting in a fine suspension. The solids were filtered, washed with water, and dried in a vacuum oven at 60° C. to afford the title compound as an off-white solid (13.1 g, 91%). ¹H NMR (400 MHz, DMSO-d₆) δ ppm 5.30 (s, 2H), 7.33-7.54 (m, 5H), 8.03 (d, J=1.77 Hz, 1H), 8.32 (d, J=1.77 Hz, 1H), 13.37 (br s, 1H); ESI-MS m/z [M+H]⁺ 308.1 (⁷⁹Br).

### Step C: 3-(benzyloxy)-5-bromo-N-(4-cyano-2,6-dimethylbenzyl)picolinamide

A mixture of 3-(benzyloxy)-5-bromopicolinic acid (12.4 g, 40.2 mmol), NMP (120 mL), 4-(aminomethyl)-3,5-dimethylbenzonitrile hydrochloride (9.50 g, 48.3 mmol) and DIPEA (35.0 mL, 201 mmol) was stirred for 30 minutes and treated with a 50 wt % solution of 2,4,6-tripropyl-1,3,5,2,4,6-trioxatriphosphorinane-2,4,6-trioxide in EtOAc (2.00 mL, 3.36 mmol). The reaction mixture was stirred for 1 hour and slowly added to water (840 mL). A solid formed and the suspension was stirred for 1 hour. The solid was filtered, washed with water and dried in a vacuum oven at 60° C. for 16 hours to give the title compound (16.94 g, 93%). ¹H NMR (400 MHz, CDCl₃) δ ppm 8.31 (d, J=1.77 Hz, 1H), 7.60 (d, J=1.77 Hz, 1H), 7.52 (br s, 1H), 7.36-7.46 (m, 5H), 7.31 (s, 2H), 5.19 (s, 2H), 4.66 (d, J=5.05 Hz, 2H), 2.36 (s, 6H); ESI-MS m/z [M+H]⁺ 450.1 (⁷⁹Br).

### Example 1: N-(4-cyanobenzyl)-3-hydroxypicolinamide

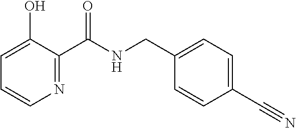 [SMILES: N#Cc1ccc(CNC(=O)c2ncccc2O)cc1]

To a 40 mL screw top vial equipped with a stir bar was charged 3-hydroxypicolinic acid (100 mg, 0.719 mmol), 1H-benzo[d][1,2,3]triazol-1-ol (126 mg, 0.935 mmol), EDC hydrochloride (179 mg, 0.935 mmol), DMF (3594 μL) and Et₃N (301 μL, 2.157 mmol). The cloudy reaction mixture was stirred for 5 minutes at room temperature. Next 4-(aminomethyl)benzonitrile (105 mg, 0.791 mmol) was added and the reaction mixture was stirred for 14 hours at room temperature. The cloudy solution was subsequently diluted with water (1 mL) and DMSO (1 mL), then purified by preparative HPLC (SunFire™ C18, 5 μm, ID 30 mm×75 mm) eluting with a gradient of 15-40% ACN (with 0.035% TFA) in H₂O (with 0.05% TFA). The product fractions were combined and lyophilized to give the title compound as a white solid (61.1 mg, 33.6%). ¹H NMR (400 MHz, DMSO-d₆) δ ppm 4.57 (d, J=6.57 Hz, 2H), 7.43 (dd, J=8.46, 1.39 Hz, 1H), 7.50-7.57 (m, 3H), 7.80 (d, J=7.78 Hz, 2 H), 8.19 (dd, J=4.29, 1.26 Hz, 1H), 9.88 (t, J=6.32 Hz, 1H), 12.36 (br s, 1H); ESI-MS m/z [M+H]⁺ 254.1.

### Example 2: N-(3-cyanobenzyl)-3-hydroxypicolinamide (Comparative Example)

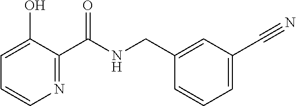 [SMILES: N#Cc1cccc(CNC(=O)c2ncccc2O)c1]

The title compound was prepared in a manner similar to Example 1, using 3-(aminomethyl)benzonitrile in place of 4-(aminomethyl)benzonitrile. ¹H NMR (400 MHz, DMSO-d₆) δ ppm 4.55 (d, J=6.57 Hz, 2H) 7.43 (d, J=8.57 Hz, 1H) 7.52-7.58 (m, 2H) 7.69 (d, J=8.08 Hz, 1H) 7.74 (d, J=7.83 Hz, 1H) 7.79 (s, 1H) 8.18 (dd, J=4.29, 1.26 Hz, 1H) 9.84 (t, J=6.32 Hz, 1H) 12.37 (br s, 1H); ESI-MS m/z [M+H]⁺ 254.1.

### Example 3: N-((6-cyanopyridin-3-yl)methyl)-3-hydroxypicolinamide

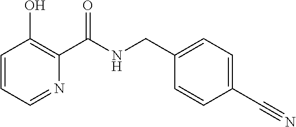 [SMILES: N#Cc1ccc(CNC(=O)c2ncccc2O)cc1]

A suspension of 3-hydroxypicolinic acid (127 mg, 0.913 mmol), 5-(aminomethyl)picolinonitrile (122 mg, 0.913 mmol), and HBTU (346 mg, 0.913 mmol) in DCM (3 mL) was treated with Et₃N (0.449 mL, 3.10 mmol). The mixture was heated at 50° C. overnight and then diluted with DCM, washed successively with 1N brine and water, dried over Na₂SO₄, and concentrated. The residue was purified using flash column chromatography on silica gel (12 g SiO₂) eluting with a gradient of 0-10% MeOH in DCM. The product-containing fractions were concentrated in vacuo and purified by HPLC, eluting with a gradient of 45-70% ACN (with 0.035% TFA) in H₂O (with 0.05% TFA) to give the title compounds as a yellow-orange solid (12.6 mg, 5.4%). ¹H NMR (400 MHz, CD₃OD) δ ppm 4.85 (s, 2H), 7.47 (dd, J=8.59, 1.52 Hz, 1H), 7.52-7.57 (m, 1H), 7.66 (dd, J=8.08, 4.80 Hz, 1H), 8.00-8.06 (m, 1H), 8.19 (dd, J=4.55, 1.52 Hz, 1H), 8.62 (dd, J=4.80, 1.52 Hz, 1H); ESI-MS m/z [M+H]⁺ 255.1.

### Example 4: N-(4-cyano-2-methylbenzyl)-3-hydroxypicolinamide

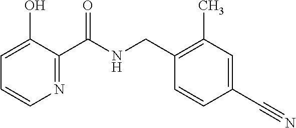 [SMILES: Cc1cc(C#N)ccc1CNC(=O)c1ncccc1O]

To a 40 mL screw top vial equipped with a stir bar was added 3-hydroxypicolinic acid (100 mg, 0.719 mmol), 1H-benzo[d][1,2,3]triazol-1-ol (126 mg, 0.935 mmol), EDC hydrochloride (179 mg, 0.935 mmol), DMF (3594 μL) and Et₃N (200 μL, 1.438 mmol). The cloudy reaction mixture was stirred for 5 minutes at room temperature. Next 4-(aminomethyl)-3-methylbenzonitrile hydrochloride (131 mg, 0.719 mmol) was added. The reaction mixture was stirred at room temperature overnight, then diluted with water (2 mL) and ethanol (2 mL), and acidified with 1N HCl to pH 5. A precipitate was collected, washed with water, and dried to give the title compound as a white solid (105.8 mg, 55.1%). ¹H NMR (400 MHz, DMSO-d₆) δ ppm 2.38 (s, 3H), 4.54 (d, J=6.32 Hz, 2H), 7.39 (d, J=7.69 Hz, 1H), 7.44 (dd, J=8.59, 1.26 Hz, 1H), 7.56 (dd, J=8.46, 4.42 Hz, 1H), 7.60-7.65 (m, 1H), 7.65-7.69 (m, 1H), 8.20 (dd, J=4.29, 1.26 Hz, 1H), 9.69-9.90 (m, 1H), 12.35 (s, 1H); ESI-MS m/z [M+H]⁺ 268.1.

### Example 5: N-(4-cyano-2-fluorobenzyl)-3-hydroxypicolinamide

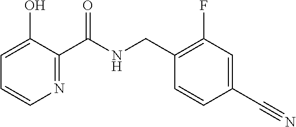 [SMILES: N#Cc1ccc(CNC(=O)c2ncccc2O)c(F)c1]

The title compared was prepared in a manner similar to Example 1, using 4-(aminomethyl)-3-fluorobenzonitrile in place of 4-(aminomethyl)benzonitrile. ¹H NMR (400 MHz, DMSO-d₆) δ ppm 4.62 (d, J=6.32 Hz, 2H), 7.46 (dd, J=8.46, 1.39 Hz, 1H), 7.53-7.60 (m, 2H), 7.69 (dd, J=8.08, 1.52 Hz, 1H), 7.87 (dd, J=10.11, 1.52 Hz, 1H), 8.21 (dd, J=4.42, 1.39 Hz, 1H), 9.79-9.90 (m, 1H), 12.26 (br s, 1H); ESI-MS m/z [M+H]⁺ 272.1.

### Example 6: N-(4-cyano-3-fluorobenzyl)-3-hydroxypicolinamide

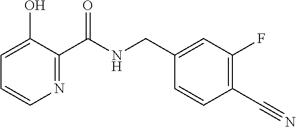 [SMILES: N#Cc1ccc(CNC(=O)c2ncccc2O)cc1F]

The title compound was prepared in a manner similar to Example 4, using 4-(aminomethyl)-2-fluorobenzonitrile hydrochloride in place of 4-(aminomethyl)-3-methylbenzonitrile hydrochloride. ¹H NMR (400 MHz, DMSO-d₆) δ ppm 4.59 (d, J=6.32 Hz, 2H), 7.32-7.39 (m, 1H), 7.40-7.50 (m, 2H), 7.56 (dd, J=8.59, 4.29 Hz, 1H), 7.89 (dd, J=7.83, 7.07 Hz, 1H), 8.19 (dd, J=4.29, 1.26 Hz, 1H), 9.89 (t, J=6.32 Hz, 1H), 12.29 (br s, 1H); ESI-MS m/z [M+H]+ 272.1.

### Example 7: N-(4-cyano-2-(trifluoromethyl)benzyl)-3-hydroxypicolinamide

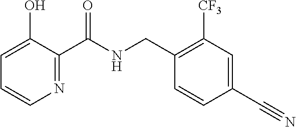 [SMILES: N#Cc1ccc(CNC(=O)c2ncccc2O)c(C(F)(F)F)c1]

The title compound was prepared in a manner similar to Example 4, using 4-(aminomethyl)-3-(trifluoromethyl)benzonitrile in place of 4-(aminomethyl)-3-methylbenzonitrile hydrochloride. 1H NMR (400 MHz, DMSO-d₆) δ ppm 4.74 (d, J=6.32 Hz, 2H), 7.46 (dd, J=8.46, 1.39 Hz, 1H), 7.59 (dd, J=8.46, 4.42 Hz, 1H), 7.68 (d, J=8.08 Hz, 1H), 8.12 (d, J=8.25 Hz, 1H), 8.23 (dd, J=4.42, 1.39 Hz, 1H), 8.29 (s, 1H), 9.93 (t, J=6.19 Hz, 1H), 12.14 (br s, 1H); ESI-MS m/z [M+H]⁺ 322.1.

### Example 8: N-(4-cyano-2-methylbenzyl)-5-(4-ethylpiperazine-1-carbonyl)-3-hydroxypicolinamide

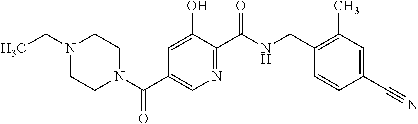 [SMILES: CCN1CCN(C(=O)c2cnc(C(=O)NCc3ccc(C#N)cc3C)c(O)c2)CC1]

To a 40 mL screw top vial charged with 3-(benzyloxy)-N-(4-cyano-2-methylbenzyl)-5-(4-ethylpiperazine-1-carbonyl)picolinamide (24 mg, 0.048 mmol) and a stir bar was added formic acid (1 mL, 26.1 mmol). The reaction mixture was stirred at 100° C. for 3 hours and then concentrated in vacuo. The resulting residue was diluted with MeOH (2 mL) and purified by preparative HPLC (SunFire™ C18, 5 μm, ID 30 mm×75 mm) eluting with a gradient of 15-40% ACN (with 0.035% TFA) in H₂O (with 0.05% TFA). The desired fractions were combined and dried under vacuum to give a TFA salt of the title compound as a light brown glass (9.4 mg, 47.8%). ¹H NMR (400 MHz, CD₃OD) δ ppm 1.27 (t, J=7.33 Hz, 3H), 2.34 (s, 3H), 2.92-3.18 (m, 4H), 3.88 (s, 6H), 4.56 (s, 2H), 7.32-7.48 (m, 4H), 8.08-8.20 (m, 1H); ESI-MS m/z [M+H]+ 408.3.

### Example 9: (R)—N-(5-cyano-2,3-dihydro-1H-inden-1-yl)-5-(4-ethylpiperazine-1-carbonyl)-3-hydroxypicolinamide

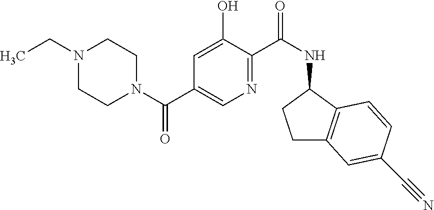 [SMILES: CCN1CCN(C(=O)c2cnc(C(=O)N[C@@H]3CCc4cc(C#N)ccc43)c(O)c2)CC1]

A TFA salt of the title compound was prepared in a manner similar to Example 8, using (R)-3-(benzyloxy)-N-(5-cyano-2,3-dihydro-1H-inden-1-yl)-5-(4-ethylpiperazine-1-carbonyl)picolinamide in place of 3-(benzyloxy)-N-(4-cyano-2-methylbenzyl)-5-(4-ethylpiperazine-1-carbonyl)picolinamide. ¹H NMR (400 MHz, CD₃OD) δ ppm 1.42 (t, J=7.33 Hz, 3H), 2.14-2.27 (m, 1H), 2.63-2.75 (m, 1H), 2.96-3.28 (m, 4H), 3.28-3.33 (m, 2H), 3.38-4.20 (m, 5H), 4.51-4.92 (m, 1H), 5.75 (br t, J=8.08 Hz, 1H), 7.47 (d, J=7.83 Hz, 1H), 7.54 (s, 1H), 7.61 (d, J=7.58 Hz, 1H), 7.69 (s, 1H), 8.20-8.28 (m, 1H); ESI-MS m/z [M+H]⁺ 420.3.

### Example 10: N-(4-cyano-2,6-dimethylbenzyl)-3-hydroxypicolinamide

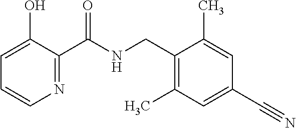 [SMILES: Cc1cc(C#N)cc(C)c1CNC(=O)c1ncccc1O]

To a 40 mL screw top vial equipped with a stir bar was added 3-hydroxypicolinic acid (100 mg, 0.719 mmol), EDC hydrochloride (179 mg, 0.935 mmol), HOBt (143 mg, 0.935 mmol), DMF (3594 μL), and Et₃N (301 μL, 2.157 mmol). The reaction mixture was stirred at room temperature for 5 minutes. Next 4-(aminomethyl)-3,5-dimethylbenzonitrile hydrochloride (170 mg, 0.863 mmol) was added. The reaction mixture was stirred at room temperature overnight, then filtered through a syringe filter, and purified by preparative HPLC (SunFire™ C18, 5 μm, ID 30 mm×75 mm) eluting with a gradient of 15-40% ACN (with 0.035% TFA) in H₂O (with 0.05% TFA). The desired fractions were combined and lyophilized to give the title compound as a white solid (100 mg, 49.5%). ¹H NMR (400 MHz, DMSO-d₆) δ ppm 2.43 (s, 6H), 4.57 (d, J=5.81 Hz, 2H), 7.41 (dd, J=8.59, 1.26 Hz, 1H), 7.52 (br d, J=4.29 Hz, 1H), 8.06-8.20 (m, 1H), 9.32 (br s, 1H), 12.37 (s, 1H); [M+H]⁺282.1.

### Example 11: N-(4-cyano-2,6-dimethylbenzyl)-3-hydroxy-5-methylpicolinamide

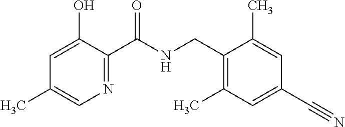 [SMILES: Cc1cnc(C(=O)NCc2c(C)cc(C#N)cc2C)c(O)c1]

The title compound was prepared in a manner similar to Example 10, using 3-hydroxy-5-methylpicolinic acid in place of 3-hydroxypicolinic acid. ¹H NMR (400 MHz, DMSO-d₆) δ ppm 2.43 (s, 6H), 4.57 (d, J=5.81 Hz, 2H), 7.41 (dd, J=8.59, 1.26 Hz, 1H), 7.48-7.51 (m, 2H), 7.52 (br d, J=4.29 Hz, 1H), 8.06-8.20 (m, 1H), 9.32 (br s, 1H), 12.27-12.44 (m, 1H); ESI-MS m/z [M+H]⁺ 296.1.

### Example 12: N-(4-cyano-2,5-dimethylbenzyl)-3-hydroxypicolinamide

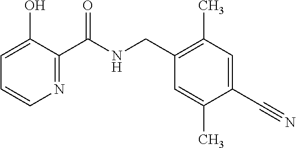 [SMILES: Cc1cc(CNC(=O)c2ncccc2O)c(C)cc1C#N]

To a 20 mL screw top vial equipped with a stir bar was added 3-hydroxypicolinic acid (50 mg, 0.359 mmol), HOBt (71.6 mg, 0.467 mmol), EDC hydrochloride (90 mg, 0.467 mmol), DMF (3594 μL) and Et₃N (200 μL, 1.438 mmol). The cloudy reaction mixture was stirred for 5 minutes at room temperature. Next 4-(aminomethyl)-2,5-dimethylbenzonitrile hydrochloride (92 mg, 0.467 mmol) was added. The reaction mixture was stirred at room temperature for 48 hours, then diluted with DMSO (1 mL) and purified by preparative HPLC (SunFire™ C18, 5 μm, ID 30 mm×75 mm) eluting with a gradient of 15-40% ACN (with 0.035% TFA) in H₂O (with 0.05% TFA). The desired fractions were combined and lyophilized to give the title compound as a white solid (6.5 mg, 6.4%). ¹H (400 MHz, CD₃OD) δ ppm 2.38 (s, 3H), 2.45 (s, 3H), 4.62 (s, 2H), 7.31 (s, 1H), 7.33-7.39 (m, 1H), 7.47 (s, 2H), 8.14 (dd, J=4.29, 1.26 Hz, 1H); ESI-MS m/z [M+H]⁺ 282.1.

### Example 13: N-(4-cyano-5-fluoro-2-methylbenzyl)-3-hydroxypicolinamide

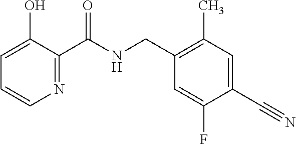 [SMILES: Cc1cc(C#N)c(F)cc1CNC(=O)c1ncccc1O]

The title compound was prepared in a manner similar to Example 12, using 4-(aminomethyl)-2-fluoro-5-methylbenzonitrile hydrochloride in place of 4-(aminomethyl)-2,5-dimethylbenzonitrile hydrochloride. ¹H (400 MHz, CD₃OD) δ ppm 2.35-2.45 (m, 3H), 4.57-4.67 (m, 2H), 7.22 (d, J=10.36 Hz, 1H), 7.36 (dd, J=8.59, 1.26 Hz, 1H), 7.46 (dd, J=8.59, 4.29 Hz, 1H), 7.56 (d, J=6.57 Hz, 1H), 8.15 (dd, J=4.29, 1.26 Hz, 1H); ESI-MS m/z [M+H]⁺ 286.1.

### Example 14: N-(4-cyano-3-fluoro-2-methylbenzyl)-3-hydroxypicolinamide

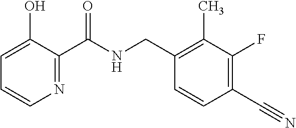 [SMILES: Cc1c(CNC(=O)c2ncccc2O)ccc(C#N)c1F]

The title compound was prepared in a manner similar to Example 12, using 4-(aminomethyl)-2-fluoro-3-methylbenzonitrile hydrochloride in place of 4-(aminomethyl)-2,5-dimethylbenzonitrile hydrochloride. ¹H (400 MHz, CD₃OD) δ ppm 2.35 (d, J=2.02 Hz, 3H), 4.63-4.70 (m, 2H), 7.29 (d, J=8.08 Hz, 1H), 7.32-7.38 (m, 1H), 7.44 (d, J=4.29 Hz, 1H), 7.54 (s, 1H), 8.14 (dd, J=4.29, 1.26 Hz, 1H); ESI-MS m/z [M+H]⁺ 286.1.

### Example 15: N-(2-chloro-4-cyanobenzyl)-3-hydroxypicolinamide

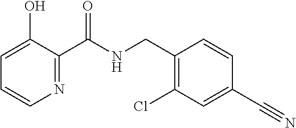 [SMILES: N#Cc1ccc(CNC(=O)c2ncccc2O)c(Cl)c1]

The title compound was prepared in a manner similar to Example 12, using 4-(aminomethyl)-3-chlorobenzonitrile hydrochloride in place of 4-(aminomethyl)-2,5-dimethylbenzonitrile hydrochloride. ¹H (400 MHz, CD₃OD) δ ppm 4.74 (s, 2H), 7.37 (d, J=8.34 Hz, 1H), 7.47 (dd, J=8.46, 4.42 Hz, 1H), 7.55 (d, J=8.08 Hz, 1H), 7.66 (dd, J=8.08, 1.52 Hz, 1H), 7.84 (d, J=1.52 Hz, 1H), 8.15 (d, J=3.79 Hz, 1H); ESI-MS m/z [M+H]⁺288.1.

### Example 16: 5-cyano-N-(4-cyanobenzyl)-3-hydroxypicolinamide

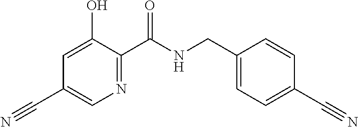 [SMILES: N#Cc1ccc(CNC(=O)c2ncc(C#N)cc2O)cc1]

A mixture of 5-cyano-N-(4-cyanobenzyl)-3-methoxypicolinamide (17 mg, 0.058 mmol) and lithium chloride (24.66 mg, 0.582 mmol) in DMA (909 μL) was stirred at room temperature for 1 hour and then at 60° C. overnight. Additional lithium chloride (24.66 mg, 0.582 mmol) and DMA (909 μL) were subsequently added and the reaction mixture was stirred at 60° C. for 72 hours and then filtered. The filtrate was purified by preparative HPLC, eluting with a gradient of 35-60% acetonitrile in water (containing formic acid) to give the title compound as an off-white solid (5.39 mg, 33.3%). ¹H NMR (400 MHz, DMSO-d₆) δ ppm 4.58 (d, J=6.06 Hz, 2H), 7.52 (d, J=8.34 Hz, 2H), 7.80 (d, J=8.08 Hz, 2H), 8.04 (s, 1H), 8.58 (br s, 1H), 10.16 (br s, 1H), 12.64 (br s, 1H); ESI-MS m/z [M+H]⁺ 279.3.

### Example 17: 5-cyano-N-(4-cyano-2-methylbenzyl)-3-hydroxypicolinamide

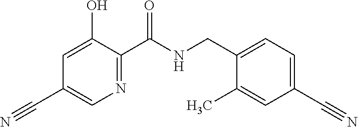 [SMILES: Cc1cc(C#N)ccc1CNC(=O)c1ncc(C#N)cc1O]

A mixture of 5-cyano-N-(4-cyano-2-methylbenzyl)-3-methoxypicolinamide (139 mg, 0.454 mmol) and lithium chloride (385 mg, 9.08 mmol) in DMA (7090 μL) was stirred at 60° C. overnight. Additional lithium chloride (385 mg, 9.08 mmol) was added and the reaction mixture was stirred at 65° C. overnight and then filtered. The filtrate was purified by preparative HPLC, eluting with a gradient of 35-60% acetonitrile in water (containing TFA) to give the title compound as an off-white solid (42 mg, 31.7%). ¹H NMR (400 MHz, DMSO-d₆) δ ppm 2.38 (s, 3H), 4.54 (d, J=6.06 Hz, 2H), 7.40 (d, J=8.08 Hz, 1H), 7.62 (dd, J=7.83, 1.26 Hz, 1H), 7.67 (d, J=1.01 Hz, 1H), 8.07 (d, J=1.52 Hz, 1H), 8.61 (d, J=1.77 Hz, 1H), 10.02 (t, J=6.19 Hz, 1H), 12.63 (s, 1H); ESI-MS m/z [M+H]⁺ 293.3.

### Example 18: N-(6-cyano-1,2,3,4-tetrahydronaphthalen-1-yl)-3-hydroxypicolinamide

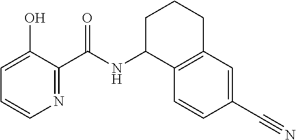 [SMILES: N#Cc1ccc2c(c1)CCCC2NC(=O)c1ncccc1O]

To a screw top vial equipped with a stir bar was added 3-hydroxypicolinic acid (83 mg, 0.6 mmol), 1H-benzo[d][1,2,3]triazol-1-ol (105 mg, 0.780 mmol), EDC hydrochloride (150 mg, 0.780 mmol), DMF (3000 μL) and Et₃N (251 μL, 1.800 mmol). The reaction mixture was stirred for 5 minutes at room temperature. Next 5-amino-5,6,7,8-tetrahydronaphthalene-2-carbonitrile hydrochloride (125 mg, 0.600 mmol) was added. The reaction mixture was stirred at room temperature for 48 hours and then filtered. The filtrate was purified by preparative HPLC, eluting with a gradient of 45-70% acetonitrile in water (containing TFA) to give the title compound as an off-white solid (10.4 mg, 5.91%). ¹H NMR (400 MHz, DMSO-d₆) δ ppm 1.74-1.84 (m, 1H), 1.91-1.99 (m, 1H), 1.99-2.05 (m, 2H), 2.77-2.85 (m, 2H), 5.25 (q, J=7.75 Hz, 1H), 7.34 (d, J=8.34 Hz, 1H), 7.41-7.47 (m, 1H), 7.51-7.59 (m, 2H), 7.62 (s, 1H), 8.15 (dd, J=4.29, 1.26 Hz, 1H), 9.50 (d, J=9.09 Hz, 1H), 12.46 (br s, 1H); ESI-MS m/z [M+H]⁺ 294.2.

### Example 19: (R)—N-(5-cyano-2,3-dihydro-1H-inden-1-yl)-3-hydroxypicolinamide

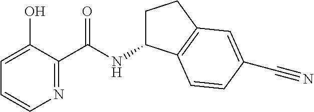 [SMILES: N#Cc1ccc2c(c1)CC[C@H]2NC(=O)c1ncccc1O]

To a screw top vial equipped with a stir bar was added 3-hydroxypicolinic acid (41.7 mg, 0.3 mmol), 1H-benzo[d][1,2,3]triazol-1-ol (52.7 mg, 0.390 mmol), EDC hydrochloride (74.8 mg, 0.390 mmol), DMF (1500 μL) and Et₃N (125 μL, 0.900 mmol). The reaction mixture was stirred for 5 minutes at room temperature. Next (R)-1-amino-2,3-dihydro-1H-indene-5-carbonitrile hydrochloride (58.4 mg, 0.300 mmol) was added. The reaction mixture was stirred at room temperature overnight and then filtered. The filtrate was purified by preparative HPLC, eluting with a gradient of 35-60% acetonitrile in water (containing TFA) to give the title compound as an off-white solid (4.37 mg, 5.22%). ¹H NMR (400 MHz, DMSO-d₆) δ ppm 2.22-2.29 (m, 1H), 2.41-2.46 (m, 1H), 2.88-2.95 (m, 1H), 3.03-3.10 (m, 1H), 5.56-5.64 (m, 1H), 7.37-7.41 (m, 1H), 7.42-7.47 (m, 1H), 7.51-7.57 (m, 1H), 7.61-7.66 (m, 1H), 7.73-7.77 (m, 1H), 8.14-8.18 (m, 1H), 9.52-9.61 (m, 1H), 12.42-12.50 (m, 1H); ESI-MS m/z [M+H]⁺ 280.2.

### Example 20: (R)—N-(5-cyano-2,3-dihydro-1H-inden-1-yl)-3-hydroxypicolinamide

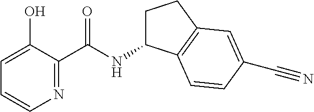 [SMILES: N#Cc1ccc2c(c1)CC[C@H]2NC(=O)c1ncccc1O]

To a screw top vial equipped with a stir bar was added 3-hydroxypicolinic acid (278 mg, 2.00 mmol), 1H-benzo[d][1,2,3]triazol-1-ol (351 mg, 2.60 mmol), EDC hydrochloride (498 mg, 2.60 mmol), DMF (10 mL) and Et₃N (836 μL, 6.00 mmol). The reaction mixture was stirred for 5 minutes at room temperature. Next (R)-1-amino-2,3-dihydro-1H-indene-5-carbonitrile hydrochloride (389 mg, 2 mmol) was added. The reaction mixture was stirred at room temperature for 96 hours, then diluted with water (5.6 mL) and ethanol (5.6 mL), acidified with 1N HCl to pH 5, and filtered. The solid and filtrate were combined, taken up in DMF and purified by preparative HPLC, eluting with 15% ACN in water (basic conditions) to give the title compound as an off-white solid (59 mg, 11%). ¹H NMR (400 MHz, DMSO-d₆) δ ppm 2.24 (dq, J=12.38, 8.93 Hz, 1H), 2.39-2.48 (m, 1H), 2.86-2.95 (m, 1H), 3.02-3.11 (m, 1H), 5.60 (q, J=8.51 Hz, 1H), 7.36-7.45 (m, 2H), 7.52 (dd, J=8.59, 4.29 Hz, 1H), 7.63 (d, J=7.83 Hz, 1H), 7.74 (s, 1H), 8.13 (dd, J=4.29, 1.01 Hz, 1H), 9.65 (d, J=8.34 Hz, 1H), 12.08-12.80 (m, 1H); ESI-MS m/z [M+H]⁺ 280.2.

### Example 21: (S)—N-(5-cyano-2,3-dihydro-1H-inden-1-yl)-3-hydroxypicolinamide

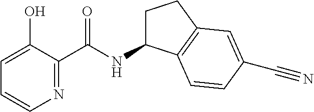 [SMILES: N#Cc1ccc2c(c1)CC[C@@H]2NC(=O)c1ncccc1O]

To a screw top vial with equipped with a stir bar was added 3-hydroxypicolinic acid (139 mg, 1 mmol), 1H-benzo[d][1,2,3]triazol-1-ol (176 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next (S)-1-amino-2,3-dihydro-1H-indene-5-carbonitrile hydrochloride (195 mg, 1.000 mmol) was added. The reaction mixture was stirred at room temperature overnight and then filtered. The filtrate was purified by preparative HPLC, eluting with a gradient of 45-70% acetonitrile in water (containing formic acid) to give the title compound as an off-white solid (52 mg, 19%). ¹H NMR (400 MHz, DMSO-d₆) δ ppm 2.20-2.30 (m, 1H), 2.42-2.48 (m, 1H), 2.91 (dt, J=16.42, 8.46 Hz, 1H), 3.03-3.11 (m, 1H), 5.60 (d, J=8.34 Hz, 1H), 7.39 (d, J=7.83 Hz, 1H), 7.44 (dd, J=8.46, 1.39 Hz, 1H), 7.51-7.57 (m, 1H), 7.63 (d, J=7.83 Hz, 1H), 7.75 (s, 1H), 8.15 (dd, J=4.29, 1.26 Hz, 1H), 9.58 (d, J=8.59 Hz, 1H), 12.46 (br s, 1H); ESI-MS m/z [M+H]⁺ 280.2.

### Example 22: N-(6-cyano-2,3-dihydrobenzofuran-3-yl)-3-hydroxypicolinamide

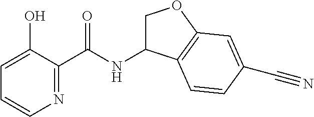 [SMILES: N#Cc1ccc2c(c1)OCC2NC(=O)c1ncccc1O]

To a screw top vial equipped with a stir bar was added 3-hydroxypicolinic acid (139 mg, 1.000 mmol), 1H-benzo[d][1,2,3]triazol-1-ol (176 mg, 1.300 mmol), EDC hydrochloride (249 mg, 1.300 mmol), DMF (5 mL) and Et₃N (418 μL, 3.00 mmol). The reaction mixture was stirred for 5 minutes at room temperature. Next 3-amino-2,3-dihydrobenzofuran-6-carbonitrile (160 mg, 1 mmol) was added. The reaction mixture was stirred at room temperature overnight and then filtered. The filtrate was purified by preparative HPLC, eluting with a gradient of 35-60% acetonitrile in water (containing formic acid) to give the title compound as an off-white solid (23 mg, 8.2%). ¹H NMR (400 MHz, DMSO-d₆) δ ppm 4.63 (dd, J=9.60, 5.81 Hz, 1H), 4.83 (t, J=9.47 Hz, 1H), 5.85-5.94 (m, 1H), 7.29-7.38 (m, 2H), 7.39-7.45 (m, 1H), 7.46-7.57 (m, 2H), 8.14 (dd, J=4.04, 1.01 Hz, 1H), 9.86 (d, J=7.07 Hz, 1H), 12.12 (br s, 1H); ESI-MS m/z [M+H]⁺ 282.0.

### Example 23: (R)—N-(5-cyano-2,3-dihydro-1H-inden-1-yl)-3-hydroxy-5-methylpicolinamide

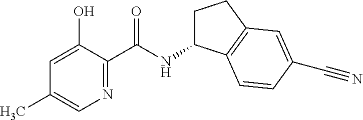 [SMILES: Cc1cnc(C(=O)N[C@@H]2CCc3cc(C#N)ccc32)c(O)c1]

To a screw top vial equipped with a stir bar was added 3-hydroxy-5-methylpicolinic acid hydrobromide (74.9 mg, 0.320 mmol), 1H-benzo[d][1,2,3]triazol-1-ol (65.4 mg, 0.416 mmol), EDC hydrochloride (80 mg, 0.416 mmol), DMF (1.6 mL) and Et₃N (178 μL, 1.280 mmol). The reaction mixture was stirred for 5 minutes at room temperature. Next (R)-1-amino-2,3-dihydro-1H-indene-5-carbonitrile hydrochloride (62.3 mg, 0.32 mmol) was added. The reaction mixture was stirred at room temperature for 72 hours and then filtered. The filtrate was purified by preparative HPLC, eluting with a gradient of 45-70% acetonitrile in water (containing formic acid) to give the title compound as an off-white solid (8 mg, 9%). ¹H NMR (400 MHz, DMSO-d₆) δ ppm 2.21-2.28 (m, 1H), 2.33 (s, 3H), 2.40-2.45 (m, 1H), 2.86-2.95 (m, 1H), 3.02-3.10 (m, 1H), 5.59 (q, J=8.42 Hz, 1H), 7.27 (dd, J=1.64, 0.88 Hz, 1H), 7.37 (d, J=7.83 Hz, 1H), 7.63 (d, J=8.84 Hz, 1H), 7.74 (s, 1H), 8.01 (d, J=1.26 Hz, 1H), 9.48 (d, J=8.59 Hz, 1H), 12.39 (s, 1H); ESI-MS m/z [M+H]+ 294.1.

### Example 24: N-((5-cyanopyridin-2-yl)methyl)-3-hydroxypicolinamide

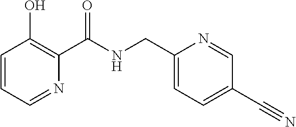 [SMILES: N#Cc1ccc(CNC(=O)c2ncccc2O)nc1]

A mixture of 6-(aminomethyl)nicotinonitrile hydrochloride (62.0 mg, 0.366 mmol), methyl 3-hydroxypicolinate (40 mg, 0.261 mmol) and DIPEA (0.091 mL, 0.522 mmol) in 2-methoxyethan-1-ol (0.8 mL) was heated in a sealed vial at 150° C. for 19 hours. The reaction mixture was subsequently purified by preparative HPLC, eluting with acetonitrile and water (with NH₄HCO₃) to give the title compound as a light yellow solid (19 mg, 28.6%). ¹H NMR (400 MHz, DMSO-d₆) δ ppm 4.64 (d, J=6.3 Hz, 2H), 7.38 (dd, J=8.5, 1.4 Hz, 1H), 7.46-7.53 (m, 2H), 8.14 (dd, J=4.4, 1.4 Hz, 1H), 8.20 (dd, J=8.2, 2.1 Hz, 1H), 8.92 (dd, J=2.0, 0.8 Hz, 1H), 9.74 (t, J=6.1 Hz, 1H), 12.22 (s, 1H); ESI-MS m/z [M+H]⁺ 255.

### Example 25: 5-chloro-N-(4-cyano-2,6-dimethylbenzyl)-3-hydroxypicolinamide

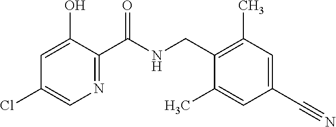 [SMILES: Cc1cc(C#N)cc(C)c1CNC(=O)c1ncc(Cl)cc1O]

A mixture of 5-chloro-N-(4-cyano-2,6-dimethylbenzyl)-3-methoxypicolinamide (77 mg, 0.233 mmol) and lithium chloride (198 mg, 4.67 mmol) in DMA (3648 μL) was stirred at 60° C. overnight and then at 80° C. overnight and filtered. The filtrate was purified by preparative HPLC, eluting with a gradient of 55-80% acetonitrile in water (containing TFA) to give the title compound as an off-white solid (28 mg, 38%). ¹H NMR (400 MHz, DMSO-d₆) δ ppm 2.42 (s, 6H), 4.56 (d, J=5.56 Hz, 2H), 7.50 (s, 2H), 7.65 (d, J=2.02 Hz, 1H), 8.16 (d, J=2.02 Hz, 1H), 9.41 (t, J=5.31 Hz, 1H), 12.63 (s, 1H); ESI-MS m/z [M+H]⁺ 316.0.

### Example 26: 5-cyano-N-(4-cyano-2,6-dimethylbenzyl)-3-hydroxypicolinamide

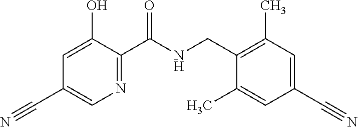 [SMILES: Cc1cc(C#N)cc(C)c1CNC(=O)c1ncc(C#N)cc1O]

A mixture of 5-cyano-N-(4-cyano-2,6-dimethylbenzyl)-3-methoxypicolinamide (12 mg, 0.037 mmol) and lithium chloride (31.8 mg, 0.749 mmol) in DMA (585 μL) was stirred at 60° C. overnight and at 80° C. overnight and then filtered. The filtrate was purified by preparative HPLC, eluting with a gradient of 40-65% acetonitrile in water (containing TFA) to give the title compound as an off-white solid (1.3 mg, 11%). ¹H NMR (400 MHz, DMSO-d₆) δ ppm 2.42 (s, 6H), 4.57 (d, J=5.31 Hz, 2H), 7.50 (s, 2H), 8.03 (d, J=1.77 Hz, 1H), 8.54 (d, J=1.77 Hz, 1H), 9.60 (t, J=5.18 Hz, 1H), 12.64 (s, 1H); ESI-MS m/z [M+H]⁺ 307.0.

### Example 27: N-(4-cyano-2,6-dimethylbenzyl)-5-fluoro-3-hydroxypicolinamide

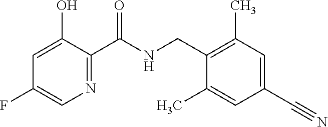 [SMILES: Cc1cc(C#N)cc(C)c1CNC(=O)c1ncc(F)cc1O]

A mixture of N-(4-cyano-2,6-dimethylbenzyl)-5-fluoro-3-methoxypicolinamide (94 mg, 0.300 mmol) and lithium chloride (254 mg, 6.00 mmol) in DMA (4688 μL) was stirred at 60° C. overnight and at 80° C. overnight and then filtered. The filtrate was purified by preparative HPLC, eluting with a gradient of 45-70% acetonitrile in water (containing TFA) to give the title compound as an off-white solid (43 mg, 48%). ¹H NMR (400 MHz, DMSO-d₆) δ ppm 2.42 (s, 6H), 4.56 (d, J=5.56 Hz, 2H), 7.43 (dd, J=10.23, 2.40 Hz, 1H), 7.49 (s, 2H), 8.15 (d, J=2.53 Hz, 1H), 9.32 (t, J=5.43 Hz, 1H), 12.75 (s, 1H); ESI-MS m/z [M+H]⁺ found 300.0.

### Example 28: N-(4-cyano-2,6-dimethylbenzyl)-3-hydroxy-5-(trifluoromethyl)picolinamide

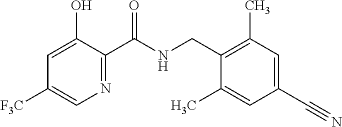 [SMILES: Cc1cc(C#N)cc(C)c1CNC(=O)c1ncc(C(F)(F)F)cc1O]

A mixture of N-(4-cyano-2,6-dimethylbenzyl)-3-methoxy-5-(trifluoromethyl)picolinamide (54 mg, 0.149 mmol) and lithium chloride (126 mg, 2.97 mmol) in DMA (2322 μL) was stirred at 80° C. overnight and then filtered. The filtrate was purified by preparative HPLC, eluting with a gradient of 55-80% acetonitrile in water (containing formic acid) to give the title compound as an off-white solid (12 mg, 23%). ¹H NMR (400 MHz, DMSO-d₆) δ ppm 2.42 (s, 6H), 4.58 (d, J=5.56 Hz, 2H), 7.50 (s, 2H), 7.85 (d, J=1.26 Hz, 1H), 8.46 (d, J=1.01 Hz, 1H), 9.59 (t, J=5.18 Hz, 1H), 12.65 (br s, 1H); ESI-MS m/z [M+H]⁺ 350.2.

### Example 29: N-(4-cyano-2,6-dimethylbenzyl)-3-hydroxy-5-methoxypicolinamide

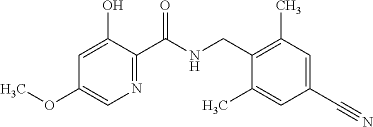 [SMILES: COc1cnc(C(=O)NCc2c(C)cc(C#N)cc2C)c(O)c1]

A mixture of N-(4-cyano-2,6-dimethylbenzyl)-3,5-dimethoxypicolinamide (104 mg, 0.320 mmol) and lithium chloride (271 mg, 6.39 mmol) in DMA (4994 μL) was stirred at 80° C. overnight and then filtered. The filtrate was purified by preparative HPLC, eluting with a gradient of 55-80% acetonitrile in water (containing formic acid) to give the title compound as an off-white solid (25 mg, 25%). ¹H NMR (400 MHz, DMSO-d₆) δ ppm 2.41-2.44 (m, 6H), 3.83-3.86 (m, 3H), 4.52-4.56 (m, 2H), 6.96-6.99 (m, 1H), 7.49-7.51 (m, 2H), 7.81-7.84 (m, 1H), 9.03-9.10 (m, 1H), 12.57-12.63 (m, 1H); ESI-MS m/z [M+H]⁺ 312.2.

### Example 30: (R)—N-(5-cyano-2,3-dihydro-1H-inden-1-yl)-5-fluoro-3-hydroxypicolinamide

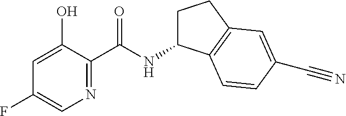 [SMILES: N#Cc1ccc2c(c1)CC[C@H]2NC(=O)c1ncc(F)cc1O]

A mixture of (R)—N-(5-cyano-2,3-dihydro-1H-inden-1-yl)-5-fluoro-3-methoxypicolinamide (51 mg, 0.164 mmol) and lithium chloride (139 mg, 3.28 mmol) in DMA (2560 μL) was stirred at 80° C. overnight and then filtered. The filtrate was purified by preparative HPLC, eluting with 50% acetonitrile in water (containing TFA) to give the title compound as an off-white solid (11 mg, 23%). ¹H NMR (400 MHz, DMSO-d₆) δ ppm 2.20-2.30 (m, 1H), 2.40-2.46 (m, 1H), 2.90 (dt, J=16.36, 8.37 Hz, 1H), 3.02-3.11 (m, 1H), 5.60 (q, J=8.34 Hz, 1H), 7.39 (d, J=7.83 Hz, 1H), 7.48 (dd, J=10.23, 2.40 Hz, 1H), 7.63 (d, J=8.34 Hz, 1H), 7.75 (s, 1H), 8.18 (d, J=2.53 Hz, 1H), 9.56 (d, J=8.59 Hz, 1H), 12.87 (br s, 1H); ESI-MS m/z [M+H]⁺ 298.0.

### Example 31: (R)-5-chloro-N-(5-cyano-2,3-dihydro-1H-inden-1-yl)-3-hydroxypicolinamide

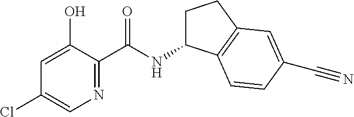 [SMILES: N#Cc1ccc2c(c1)CC[C@H]2NC(=O)c1ncc(Cl)cc1O]

A mixture of (R)-5-chloro-N-(5-cyano-2,3-dihydro-1H-inden-1-yl)-3-methoxypicolinamide (34 mg, 0.104 mmol) and lithium chloride (88 mg, 2.075 mmol) in DMA (1621 μL) was stirred at 80° C. overnight and then filtered. The filtrate was purified by preparative HPLC, eluting with a gradient of 55-80% acetonitrile in water (containing formic acid) to give the title compound as an off-white solid (6 mg, 18%). ¹H NMR (400 MHz, DMSO-d₆) δ ppm 2.24 (dq, J=12.57, 8.86 Hz, 1H), 2.38-2.47 (m, 1H), 2.90 (dt, J=16.48, 8.31 Hz, 1H), 3.00-3.11 (m, 1H), 5.59 (q, J=8.34 Hz, 1H), 7.39 (d, J=7.83 Hz, 1H), 7.63 (d, J=7.83 Hz, 1H), 7.66-7.78 (m, 2H), 8.18 (d, J=2.02 Hz, 1H), 9.65 (d, J=8.59 Hz, 1H), 12.72 (br s, 1H); ESI-MS m/z [M+H]⁺ 314.0.

### Example 32: (R)—N-(5-cyano-2,3-dihydro-1H-inden-1-yl)-3-hydroxy-5-(trifluoromethyl)picolinamide

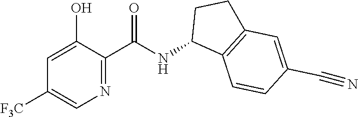 [SMILES: N#Cc1ccc2c(c1)CC[C@H]2NC(=O)c1ncc(C(F)(F)F)cc1O]

A mixture of (R)—N-(5-cyano-2,3-dihydro-1H-inden-1-yl)-3-methoxy-5-(trifluoromethyl)picolinamide (68 mg, 0.188 mmol) and lithium chloride (160 mg, 3.76 mmol) in DMA (2941 μL) was stirred at 80° C. overnight and then filtered. The filtrate was purified by preparative HPLC, eluting with a gradient of 55-80% acetonitrile in water (containing formic acid) to give the title compound as an off-white solid (25 mg, 38%). ¹H NMR (400 MHz, DMSO-d₆) δ ppm 2.20-2.35 (m, 1H), 2.45 (br s, 1H), 2.91 (dt, J=15.92, 7.96 Hz, 1H), 3.03-3.14 (m, 1H), 5.62 (d, J=8.08 Hz, 1H), 7.41 (d, J=7.58 Hz, 1H), 7.64 (d, J=7.58 Hz, 1H), 7.75 (br s, 1H), 7.87 (br s, 1H), 8.47 (br s, 1H), 9.88 (br s, 1H), 12.77 (br s, 1H); ESI-MS m/z [M+H]⁺ 348.0.

### Example 33: (R)—N-(5-cyano-2,3-dihydro-1H-inden-1-yl)-3-hydroxy-5-methoxypicolinamide

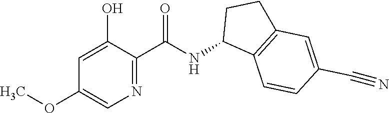 [SMILES: COc1cnc(C(=O)N[C@@H]2CCc3cc(C#N)ccc32)c(O)c1]

A mixture of (R)—N-(5-cyano-2,3-dihydro-1H-inden-1-yl)-3,5-dimethoxypicolinamide (97 mg, 0.30 mmol) and lithium chloride (254 mg, 6.00 mmol) in DMA (4687 μL) was stirred at 80° C. overnight and then filtered. The filtrate was purified by preparative HPLC, eluting with a gradient of 45-70% acetonitrile in water (containing TFA) to give the title compound as an off-white solid (15 mg, 16%). ¹H NMR (400 MHz, DMSO-d₆) δ ppm 2.23 (dq, J=12.51, 8.97 Hz, 1H), 2.40-2.48 (m, 1H), 2.89 (dt, J=16.42, 8.46 Hz, 1H), 3.01-3.09 (m, 1H), 3.87 (s, 3H), 5.58 (q, J=8.34 Hz, 1H), 7.01 (d, J=2.53 Hz, 1H), 7.37 (d, J=7.83 Hz, 1H), 7.63 (d, J=7.83 Hz, 1H), 7.74 (s, 1H), 7.85 (d, J=2.53 Hz, 1H), 9.30 (d, J=8.59 Hz, 1H), 12.71 (br s, 1H); ESI-MS m/z [M+H]⁺ 310.2.

### Example 34: (R)-5-cyano-N-(5-cyano-2,3-dihydro-1H-inden-1-yl)-3-hydroxypicolinamide

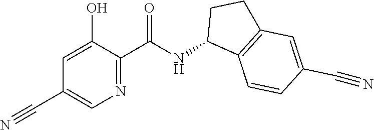 [SMILES: N#Cc1cnc(C(=O)N[C@@H]2CCc3cc(C#N)ccc32)c(O)c1]

A mixture of (R)-5-cyano-N-(5-cyano-2,3-dihydro-1H-inden-1-yl)-3-methoxypicolinamide (68 mg, 0.214 mmol) and lithium chloride (181 mg, 4.27 mmol) in DMA (3338 μL) was stirred at 80° C. overnight and filtered. The filtrate was purified by preparative HPLC, eluting with a gradient of 45-70% acetonitrile in water (containing formic acid) to give the title compound as an off-white solid (1 mg, 2%). ¹H NMR (400 MHz, CD₃OD) δ ppm 2.08-2.24 (m, 1H), 2.57-2.69 (m, 1H), 2.99 (dt, J=16.42, 8.46 Hz, 1H), 3.09-3.20 (m, 1H), 5.64-5.76 (m, 1H), 7.43 (d, J=7.83 Hz, 1H), 7.56 (d, J=7.58 Hz, 1H), 7.64 (s, 1H), 7.78 (br s, 1H), 8.38 (br s, 1H); ESI-MS m/z [M+H]⁺ 305.0.

### Example 35: N-(4-cyano-2-methylbenzyl)-5-fluoro-3-hydroxypicolinamide

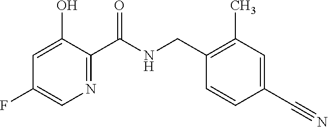 [SMILES: Cc1cc(C#N)ccc1CNC(=O)c1ncc(F)cc1O]

A mixture of N-(4-cyano-2-methylbenzyl)-5-fluoro-3-methoxypicolinamide (20 mg, 0.067 mmol) and lithium chloride (56.7 mg, 1.336 mmol) in DMA (1044 μL) was stirred at 80° C. overnight and then filtered. The filtrate was purified by preparative HPLC, eluting with a gradient of 45-70% acetonitrile in water (containing TFA) to give the title compound as an off-white solid (5 mg, 26.2% yield). ¹H NMR (400 MHz, DMSO-d₆) δ ppm 2.38 (s, 3H), 4.53 (d, J=6.32 Hz, 2H), 7.38 (d, J=8.08 Hz, 1H), 7.48 (dd, J=10.36, 2.27 Hz, 1H), 7.62 (d, J=8.08 Hz, 1H), 7.66 (s, 1H), 8.23 (d, J=2.27 Hz, 1H), 9.77 (t, J=6.06 Hz, 1H), 12.73 (br s, 1H); ESI-MS m/z [M+H]⁺ 286.1.

### Example 36: 5-chloro-N-(4-cyano-2-methylbenzyl)-3-hydroxypicolinamide

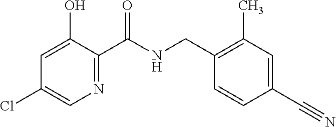 [SMILES: Cc1cc(C#N)ccc1CNC(=O)c1ncc(Cl)cc1O]

A mixture of 5-chloro-N-(4-cyano-2-methylbenzyl)-3-methoxypicolinamide (44 mg, 0.139 mmol) and lithium chloride (118 mg, 2.79 mmol) in DMA (2177 t.L) was stirred at 80° C. overnight and then filtered. The filtrate was purified by preparative HPLC, eluting with a gradient of 45-70% acetonitrile in water (containing TFA) to give the title compound as an off-white solid (16 mg, 38%). ¹H NMR (400 MHz, DMSO-d₆) δ ppm 2.38 (s, 3H), 4.53 (d, J=6.32 Hz, 2H), 7.38 (d, J=7.83 Hz, 1H), 7.62 (dd, J=8.08, 1.26 Hz, 1H), 7.66 (s, 1H), 7.69 (d, J=2.02 Hz, 1H), 8.24 (d, J=2.02 Hz, 1H), 9.85 (t, J=6.19 Hz, 1H), 12.60 (br s, 1H); ESI-MS m/z [M+H]⁺ 302.1.

### Example 37: N-(4-cyano-2-methylbenzyl)-3-hydroxy-5-(trifluoromethyl)picolinamide

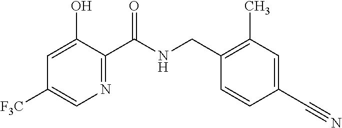 [SMILES: Cc1cc(C#N)ccc1CNC(=O)c1ncc(C(F)(F)F)cc1O]

A mixture of N-(4-cyano-2-methylbenzyl)-3-methoxy-5-(trifluoromethyl)picolinamide (51 mg, 0.146 mmol) and lithium chloride (124 mg, 2.92 mmol) in DMA (2281 μL) was stirred at 80° C. overnight and then filtered. The filtrate was purified by preparative HPLC, eluting with a gradient of 45-70% acetonitrile in water (containing TFA) to give the title compound as an off-white solid (18 mg, 37%). ¹H NMR (400 MHz, DMSO-d₆) δ ppm 2.38 (s, 3H), 4.55 (d, J=6.32 Hz, 2H), 7.41 (d, J=8.08 Hz, 1H), 7.62 (dd, J=7.96, 1.39 Hz, 1H), 7.67 (s, 1H), 7.90 (d, J=1.26 Hz, 1H), 8.54 (d, J=1.01 Hz, 1H), 10.01 (t, J=6.19 Hz, 1H), 12.66 (br s, 1H); ESI-MS m/z [M+H]⁺ 336.2.

### Example 38: N-(4-cyano-2-methylbenzyl)-3-hydroxy-5-methoxypicolinamide

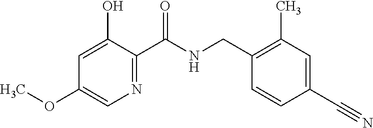 [SMILES: COc1cnc(C(=O)NCc2ccc(C#N)cc2C)c(O)c1]

A mixture of N-(4-cyano-2-methylbenzyl)-3,5-dimethoxypicolinamide (67 mg, 0.215 mmol) and lithium chloride (182 mg, 4.30 mmol) in DMA (3363 μL) was stirred at 80° C. overnight and then filtered. The filtrate was purified by preparative HPLC, eluting with a gradient of 45-70% acetonitrile in water (containing TFA) to give the title compound as an off-white solid (22 mg, 34%). ¹H NMR (400 MHz, DMSO-d₆) δ ppm 2.37 (s, 3H), 3.87 (s, 3H), 4.51 (d, J=6.32 Hz, 2H), 7.00 (d, J=2.53 Hz, 1H), 7.36 (d, J=7.83 Hz, 1H), 7.62 (dd, J=7.96, 1.39 Hz, 1H), 7.66 (s, 1H), 7.86-7.93 (m, 1H), 9.54 (t, J=6.19 Hz, 1H), 12.58 (br s, 1H); ESI-MS m/z [M+H]⁺ 298.2.

### Example 39: N-(1-(4-cyanophenyl)ethyl)-3-hydroxypicolinamide

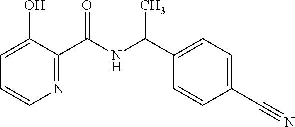 [SMILES: CC(NC(=O)c1ncccc1O)c1ccc(C#N)cc1]

To a stirring solution of 3-hydroxypicolinic acid (0.132 g, 0.949 mmol), 1H-benzo[d][1,2,3]triazol-1-ol (0.192 g, 1.423 mmol), EDC hydrochloride (0.273 g, 1.423 mmol), and N-ethyl-N-isopropylpropan-2-amine (0.992 mL, 5.69 mmol) in DMF (2 mL) was added 4-(1-aminoethyl)benzonitrile hydrochloride (0.260 g, 1.423 mmol). The reaction mixture was heated to 50° C. for 7 hours, then cooled to room temperature, diluted with water (6 mL), acidified to pH 5.5 with 1M HCl, and extracted with EtOAc (1×20 mL, 2×10 mL). The organic layers were combined, washed with brine (10 mL), dried over Na₂SO₄ and concentrated to give an oil, which was purified by preparative HPLC (SunFire™ C18, 5 μm, ID 30 mm×75 mm) eluting with a gradient of 15-40% ACN (with 0.035% TFA) in H₂O (with 0.05% TFA). The pure fractions were combined and concentrated to give the title compound as a viscous orange oil (91 mg, 36%). ¹H NMR (400 MHz, DMSO-d₆) δ ppm 1.56 (d, J=7.07 Hz, 3H), 5.17-5.32 (m, 1H), 7.41 (dd, J=8.46, 1.39 Hz, 1H), 7.55 (dd, J=8.46, 4.42 Hz, 1H), 7.61-7.66 (m, 2H), 7.77-7.84 (m, 2H), 8.20 (dd, J=4.42, 1.39 Hz, 1H), 9.68 (d, J=8.34 Hz, 1H), 12.33 (br s, 1H); ESI-MS m/z [M+H]+ 268.1.

### Example 40: N-(4-cyano-2,6-dimethylbenzyl)-3-hydroxy-5-((3-methoxypropyl)amino)picolinamide

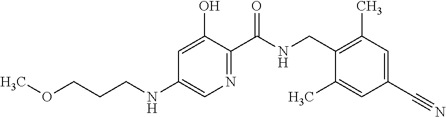 [SMILES: COCCCNc1cnc(C(=O)NCc2c(C)cc(C#N)cc2C)c(O)c1]

### Step A: 3-(benzyloxy)-N-(4-cyano-2,6-dimethylbenzyl)-5-((3-methoxypropyl)amino)picolinamide

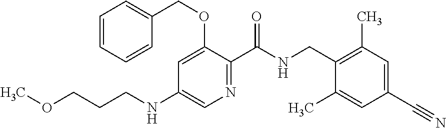 [SMILES: COCCCNc1cnc(C(=O)NCc2c(C)cc(C#N)cc2C)c(OCc2ccccc2)c1]

To a solution of 3-(benzyloxy)-5-bromo-N-(4-cyano-2,6-dimethylbenzyl)picolinamide (16.89 g, 37.5 mmol) in 1,4-dioxane (125 mL) was added sodium tert-butoxide (9.01 g, 94 mmol). The mixture was degassed by bubbling nitrogen through the suspension for 3 minutes. BrettPhos Pd G3 (3.40 g, 3.75 mmol) and 3-methoxypropan-1-amine (8.43 mL, 83 mmol) were added. The reaction mixture was degassed again and stirred in a closed system at 110° C. for 3 hours. Following reaction the mixture was partitioned between water (300 mL) and isopropyl acetate (300 mL). The layers were split and the organic layer was held in reserve. The aqueous phase was extracted with isopropyl acetate (2×150 mL). The organic layers were combined, washed with saturated NaCl (aq), dried over Na₂SO₄, filtered and concentrated. The concentrate was purified by flash column chromatography on silica gel, eluting with a gradient of 0-5% methanol in dichloromethane to give the title compound as a solid (6.3 g, 37%). ESI-MS m/z [M+H]⁺459.4.

### Step B: N-(4-cyano-2,6-dimethylbenzyl)-3-hydroxy-5-((3-methoxypropyl)amino)picolinamide

A solution of 3-(benzyloxy)-N-(4-cyano-2,6-dimethylbenzyl)-5-((3-methoxypropyl)amino)picolinamide (6.3 g, 13.74 mmol) in THF (70 mL) was degassed with nitrogen. To the solution was added 20% palladium hydroxide on carbon (1.929 g, 2.75 mmol). The reaction mixture was treated with hydrogen (1 atm via balloon) at room temperature for 2 hours. Following hydrogenolysis, the reaction mixture was filtered through a pad of Celite® which was washed with THF (50 mL). The filtrate was concentrated and the crude product was purified by flash column chromatography on silica gel, eluting with a gradient of 0-50% EtOAc in DCM. The pure fractions were combined and concentrated to give the title compound as a solid (4.07 g, 80%). ¹H NMR (500 MHz, CD₃CN) δ ppm 1.79-1.85 (m, 2H), 2.45 (s, 6H), 3.16-3.21 (m, 2H), 3.30 (s, 3H), 3.45 (t, J=6.10 Hz, 2H), 4.62 (d, J=5.61 Hz, 2H), 5.22 (m, 1H), 6.30 (d, J=2.44 Hz, 1H), 7.42 (s, 2H), 7.45 (d, J=2.44 Hz, 1H), 7.80 (m, 1H), 12.17 (s, 1H); ESI-MS m/z [M+H]⁺ 369.3.

### Example: 41: N-(4-cyano-2,6-dimethylbenzyl)-5-(4-ethylpiperazin-1-yl)-3-hydroxypicolinamide

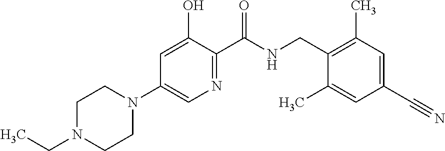 [SMILES: CCN1CCN(c2cnc(C(=O)NCc3c(C)cc(C#N)cc3C)c(O)c2)CC1]

### Step A: 3-(benzyloxy)-N-(4-cyano-2,6-dimethylbenzyl)-5-(4-ethylpiperazin-1-yl)picolinamide

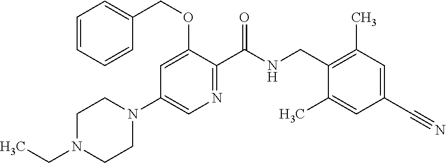 [SMILES: CCN1CCN(c2cnc(C(=O)NCc3c(C)cc(C#N)cc3C)c(OCc3ccccc3)c2)CC1]

A solution of 3-(benzyloxy)-5-bromo-N-(4-cyano-2,6-dimethylbenzyl)picolinamide (102 mg, 0.227 mmol) in toluene (1 mL) was degassed with nitrogen for 2 minutes. To the solution was added cesium carbonate (148 mg, 0.453 mmol), Pd₂(dba)₃ (10.37 mg, 0.011 mmol) and rac-BINAP (14.10 mg, 0.023 mmol) all under a nitrogen atmosphere. To the stirring suspension was added 1-ethylpiperazine (0.035 mL, 0.272 mmol). The reaction mixture was heated to 100° C. in a closed system for 1 hour, then cooled to room temperature, diluted with EtOAc (20 mL), and washed successively with water (10 mL) and saturated NH₄Cl (aq) (10 mL). The organic phase was dried over MgSO₄, filtered, and concentrated to give an oil, which was used without further purification. ESI-MS m/z [M+H]⁺ 484.3.

### Step B: N-(4-cyano-2,6-dimethylbenzyl)-5-(4-ethylpiperazin-1-yl)-3-hydroxypicolinamide

A solution of 3-(benzyloxy)-N-(4-cyano-2,6-dimethylbenzyl)-5-(4-ethylpiperazin-1-yl)picolinamide (111 mg, 0.230 mmol) in THF (3 mL) was degassed with nitrogen. To the solution was added 20% palladium hydroxide on carbon (32.2 mg, 0.046 mmol). The reaction mixture was treated with hydrogen (1 atm via balloon) at room temperature for 30 minutes. Following hydrogenolysis, the reaction mixture was filtered. The filtrate was concentrated to give a crude yellow oil, which was purified by preparative HPLC (SunFire™ C18, 5 μm, ID 30 mm×75 mm) eluting with a gradient of 15-40% ACN (with 0.035% TFA) in H₂O (with 0.05% TFA) to give a TFA salt of the title compound (22 mg, 18.5%). 1H NMR (400 MHz, DMSO-d₆) δ ppm ¹H NMR (500 MHz, DMSO-d₆) δ ppm 1.01 (t, J=7.32 Hz, 3H), 2.33 (q, J=7.24 Hz, 2H), 2.44 (s, 8H), 2.52 (m, 2H), 2.96-3.06 (m, 4H), 4.48 (d, J=5.37 Hz, 2H), 5.76 (d, J=0.98 Hz, 1H), 5.97 (br s, 1H), 7.19-7.24 (m, 1H), 7.49 (s, 2H) 12.71 (br s, 1H); ESI-MS m/z [M+H]⁺ 394.3.

### Example 42: N-(4-cyano-2,6-dimethylbenzyl)-5-(4-(2,2-difluoroethyl)piperazin-1-yl)-3-hydroxypicolinamide

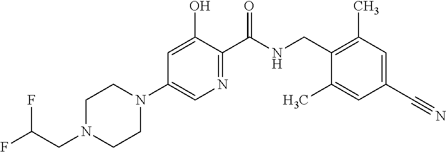 [SMILES: Cc1cc(C#N)cc(C)c1CNC(=O)c1ncc(N2CCN(CC(F)F)CC2)cc1O]

### Step A: 3-(benzyloxy)-N-(4-cyano-2,6-dimethylbenzyl)-5-(4-(2,2-difluoroethyl)piperazin-1-yl)picolinamide

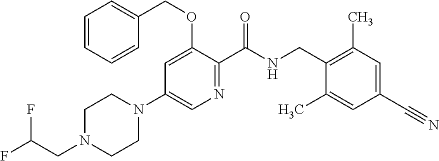 [SMILES: Cc1cc(C#N)cc(C)c1CNC(=O)c1ncc(N2CCN(CC(F)F)CC2)cc1OCc1ccccc1]

A solution of 3-(benzyloxy)-5-bromo-N-(4-cyano-2,6-dimethylbenzyl)picolinamide (126 mg, 0.280 mmol) in toluene (1 mL) was degassed with nitrogen. To the solution was added cesium carbonate (283 mg, 0.867 mmol), Pd₂(dba)₃ (12.81 mg, 0.014 mmol) and rac-BINAP (17.42 mg, 0.028 mmol) under nitrogen. To the stirring suspension was added 1-(2,2-difluoroethyl)piperazine hydrochloride (57.4 mg, 0.308 mmol). The reaction mixture was heated to 100° C. in a closed system for 1 hour, then cooled, diluted with EtOAc (20 mL), and washed successively with water (10 mL) and saturated NH₄Cl (aq) (10 mL). The organic phase was dried over MgSO₄, filtered, and concentrated. The resulting crude oil was used without further purification. ESI-MS m/z [M+H]⁺ 520.3.

### Step B: N-(4-cyano-2,6-dimethylbenzyl)-5-(4-(2,2-difluoroethyl)piperazin-1-yl)-3-hydroxypicolinamide

A solution of 3-(benzyloxy)-N-(4-cyano-2,6-dimethylbenzyl)-5-(4-(2,2-difluoroethyl)piperazin-1-yl)picolinamide (145 mg, 0.279 mmol) and ethylenediamine (0.5 M solution in THF, 0.112 mL, 0.056 mmol) in THF (3 mL) was degassed with nitrogen. To the solution was added 10% palladium on carbon (50% wet Degussa® type) (59.4 mg, 0.028 mmol) under a nitrogen atmosphere. The reaction mixture was treated with hydrogen (1 atm via balloon) for 16 hours. Following hydrogenolysis, the reaction mixture was filtered. The filtrate was concentrated to give a yellow oil, which was purified by preparative HPLC (SunFire™ C18, 5 μm, ID 30 mm×75 mm) eluting with a gradient of 15-40% ACN (with 0.035% TFA) in H₂O (with 0.05% TFA) to give as TFA salt of the title compound as a solid (53 mg, 35%). ¹H NMR (400 MHz, DMSO-d₆) δ ppm ¹H NMR (500 MHz, DMSO-d₆) δ ppm 2.42 (s, 6H), 2.58-2.68 (m, 4H), 2.72-2.84 (m, 2H), 3.24-3.33 (m, 4H), 4.52 (d, J=5.61 Hz, 2H), 5.96-6.35 (m, 1H), 6.63 (s, 1H), 7.49 (s, 2H), 7.81 (s, 1H), 8.94 (br s, 1H), 12.32 (s, 1H); ESI-MS m/z [M+H]⁺ 430.3.

Table 1 lists biological data for some of the compounds shown in the examples, where larger pIC₅₀ and pEC₅₀ values represent higher potency or activity, respectively. The IC₅₀ data (reported in Table 1 as pIC₅₀) were obtained using the enzyme-based assay described in the specification under the heading “Inhibition of PHD2 Enzyme.” The EC₅₀ data (reported in Table 1 as pEC₅₀) were obtained using the cell-based assay described in the specification under the heading “Cell-based HIF-α Stabilization Assay.”

TABLE 1
Inhibition of PHD2 (pIC₅₀) and Stabilization of HIF-α (pEC₅₀)

| Example | pIC₅₀ | pEC₅₀ |
|---|---|---|
| 1 | 7.2 | 5.0 |
| 2 | 5.5 | — |
| 3 | 6.0 | — |
| 4 | 7.7 | 5.1 |
| 5 | 7.5 | 5.1 |
| 6 | 7.5 | 5.2 |
| 7 | 5.4 | — |
| 8 | 7.0 | 4.9 |
| 9 | 7.3 | 4.9 |
| 10 | 7.9 | 5.5 |
| 11 | 7.5 | 5.1 |
| 12 | 7.4 | 4.6 |
| 13 | 7.8 | 5.0 |
| 14 | 7.9 | 4.9 |
| 15 | 7.6 | 4.9 |
| 16 | 6.6 | 5.2 |
| 17 | 6.5 | 4.3 |
| 18 | 5.9 | 4.3 |
| 19 | 8.0 | 5.2 |
| 20 | 7.9 | 5.2 |
| 21 | 5.6 | 4.3 |
| 22 | 7.4 | 5.1 |
| 23 | 7.5 | 5.1 |
| 24 | 6.4 | 5.1 |
| 25 | 7.7 | 4.9 |
| 26 | 7.6 | 4.9 |
| 27 | 7.8 | 5.2 |
| 28 | 7.3 | 4.8 |
| 29 | 8.0 | 5.8 |
| 30 | 7.8 | 4.8 |
| 31 | 7.5 | 4.6 |
| 32 | 6.9 | 4.4 |
| 33 | 8.1 | 5.2 |
| 34 | 7.4 | 4.9 |
| 35 | 7.7 | 4.6 |
| 36 | 7.4 | 4.6 |
| 37 | 6.6 | — |
| 38 | 8.0 | 5.1 |
| 39 | 5.8 | 4.3 |
| 40 | 7.5 | 5.6 |
| 41 | 7.1 | 5.2 |
| 42 | 7.4 | 5.4 |

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references cited in the disclosure, including patents, patent applications and publications, are herein incorporated by reference in their entirety and for all purposes.

1. A compound of Formula 1, 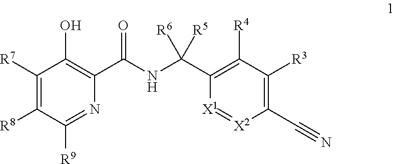
or a pharmaceutically acceptable salt thereof in which:
X¹ is selected from N and CR¹, and
X² is selected from N and CR², provided:
(a) no more than one of X¹ and X² is N, and
(b) when R⁴ and R⁵ are linked to form an ethane-1,2-diyl, a propane-1,3-diyl or a methane-1,1-diyloxy, then X¹ is CR¹ and X² is CR², and
(c) when X¹ is CR¹ and X² is CR², then at least one of R¹, R², R³, R⁴, R⁵, R⁶, R⁷, R⁸, and R⁹ is not hydrogen;
R¹, R², and R³ are each independently selected from hydrogen, halo, and C₁₋₄ alkyl optionally substituted with from one to three halo substituents;
R⁴ is selected from hydrogen, halo, and C₁₋₄ alkyl optionally substituted with from one to three halo substituents, and
R⁵ is selected from hydrogen and C₁₋₄ alkyl, or
R⁴ and R⁵ are linked to form an ethane-1,2-diyl, a propane-1,3-diyl or a methane-1,1-diyloxy;
R⁶ is selected from hydrogen and C₁₋₄ alkyl;
R⁷, R⁸, and R⁹ are each independently selected from hydrogen, halo, cyano, —N(R^{a})R^{b}, —C(O)N(R^{a})R^{b}, —OR, and C₁₋₄ alkyl optionally substituted with from one to three halo substituents, wherein:
R^{a} and R^{b} are each independently selected from hydrogen and C₁₋₄ alkyl optionally substituted with from one to three substituents independently selected from halo and —OR^{d}, or
R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has one or two heteroatoms as ring members in which one of the heteroatoms is nitrogen and another of the heteroatoms, if present, is independently selected from N, O, and S;
R^{c} is selected from hydrogen and C₁₋₄ alkyl optionally substituted with from one to three substituents independently selected from halo and —OR^{d}; and
R^{d} is selected from hydrogen and C₁₋₄ alkyl.

2-3. (canceled)

4. The compound or pharmaceutically acceptable salt according to claim 1, wherein X¹ is CR¹ and X² is CR².

5. The compound or pharmaceutically acceptable salt according to claim 1, wherein R¹, R², and R³ are each independently selected from hydrogen, halo, and methyl.

6. (canceled)

7. The compound or pharmaceutically acceptable salt according to claim 1, wherein R⁴ is selected from hydrogen, halo, and methyl.

8. (canceled)

9. The compound or pharmaceutically acceptable salt according to claim 1, wherein R⁵ is selected from hydrogen and methyl.

10-11. (canceled)

12. The compound or pharmaceutically acceptable salt according to claim 1, wherein R⁶ is hydrogen.

13. The compound or pharmaceutically acceptable salt according to claim 1, wherein R⁴ and R⁵ are linked to form an ethane-1,2-diyl.

14. The compound or pharmaceutically acceptable salt according to claim 1, wherein R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
R^{a} and R^{b} are each independently selected from hydrogen and C₁₋₄ alkyl optionally substituted with from one to three substituents independently selected from halo and —OR^{d}, or
R^{a} and R^{b} together with the nitrogen atom to which they are attached form a C₃₋₅ heterocyclyl optionally substituted with C₁₋₄ alkyl, wherein the C₁₋₄ alkyl substituent is optionally substituted with from one to three halo substituents, and the C₃₋₅ heterocyclyl moiety has 5 or 6 ring members and one or two heteroatoms as ring members in which one of the heteroatoms is nitrogen and another of the heteroatoms, if present, is independently selected from N, O, and S;
R^{c} is selected from hydrogen and C₁₋₄ alkyl optionally substituted with from one to three substituents independently selected from halo and —OR^{d}; and
R^{d} is selected from hydrogen and C₁₋₄ alkyl.

15. (canceled)

16. The compound or pharmaceutically acceptable salt according to claim 1, wherein R⁷, R⁸, and R⁹ are each independently selected from hydrogen, halo, cyano, —N(R^{a})R^{b}, —C(O)N(R^{a})R^{b}, —OR^{c}, and C₁₋₄ alkyl optionally substituted with from one to three halo substituents, wherein:
R^{a} and R^{b} are each independently selected from hydrogen and C₁₋₄ alkyl optionally substituted with from one to three substituents independently selected from halo and —OR^{d}, or
R^{a} and R^{b} together with the nitrogen atom to which they are attached form a piperazine-1-yl optionally substituted with C₁₋₄ alkyl, wherein the C₁₋₄ alkyl substituent is optionally substituted with from one to three halo substituents;
R^{c} is selected from hydrogen and C₁₋₄ alkyl optionally substituted with from one to three substituents independently selected from halo and —OR^{d}; and
R^{d} is selected from hydrogen and C₁₋₄ alkyl.

17. The compound or pharmaceutically acceptable salt according to claim 1, wherein R⁷ is selected from hydrogen, halo, cyano, and C₁₋₄ alkyl.

18. (canceled)

19. The compound or pharmaceutically acceptable salt according to claim 1, wherein R⁸ is selected from hydrogen, halo, cyano, —N(R^{a})R^{b}, —C(O)N(R^{a})R^{b}, —OR^{c}, methyl, and —CF₃.

20. (canceled)

21. The compound or pharmaceutically acceptable salt according to claim 1, wherein R⁹ is selected from hydrogen, halo, cyano, and C₁₋₄ alkyl.

22. (canceled)

23. The compound according to claim 1, which is selected from the following compounds:
N-((6-cyanopyridin-3-yl)methyl)-3-hydroxypicolinamide;
N-(4-cyano-2-methylbenzyl)-3-hydroxypicolinamide;
N-(4-cyano-2-fluorobenzyl)-3-hydroxypicolinamide;
N-(4-cyano-3-fluorobenzyl)-3-hydroxypicolinamide;
N-(4-cyano-2-(trifluoromethyl)benzyl)-3-hydroxypicolinamide;
N-(4-cyano-2-methylbenzyl)-5-(4-ethylpiperazine-1-carbonyl)-3-hydroxypicolinamide;
(R)—N-(5-cyano-2,3-dihydro-1H-inden-1-yl)-5-(4-ethylpiperazine-1-carbonyl)-3-hydroxypicolinamide;
N-(4-cyano-2,6-dimethylbenzyl)-3-hydroxypicolinamide;
N-(4-cyano-2,6-dimethylbenzyl)-3-hydroxy-5-methylpicolinamide;
N-(4-cyano-2,5-dimethylbenzyl)-3-hydroxypicolinamide;
N-(4-cyano-5-fluoro-2-methylbenzyl)-3-hydroxypicolinamide;
N-(4-cyano-3-fluoro-2-methylbenzyl)-3-hydroxypicolinamide;
N-(2-chloro-4-cyanobenzyl)-3-hydroxypicolinamide;
5-cyano-N-(4-cyanobenzyl)-3-hydroxypicolinamide;
5-cyano-N-(4-cyano-2-methylbenzyl)-3-hydroxypicolinamide;
N-(6-cyano-1,2,3,4-tetrahydronaphthalen-1-yl)-3-hydroxypicolinamide;
(R)—N-(5-cyano-2,3-dihydro-1H-inden-1-yl)-3-hydroxypicolinamide;
(S)—N-(5-cyano-2,3-dihydro-1H-inden-1-yl)-3-hydroxypicolinamide;
N-(6-cyano-2,3-dihydrobenzofuran-3-yl)-3-hydroxypicolinamide;
(R)—N-(5-cyano-2,3-dihydro-1H-inden-1-yl)-3-hydroxy-5-methylpicolinamide;
N-((5-cyanopyridin-2-yl)methyl)-3-hydroxypicolinamide;
5-chloro-N-(4-cyano-2,6-dimethylbenzyl)-3-hydroxypicolinamide;
5-cyano-N-(4-cyano-2,6-dimethylbenzyl)-3-hydroxypicolinamide;
N-(4-cyano-2,6-dimethylbenzyl)-5-fluoro-3-hydroxypicolinamide;
N-(4-cyano-2,6-dimethylbenzyl)-3-hydroxy-5-(trifluoromethyl)picolinamide;
N-(4-cyano-2,6-dimethylbenzyl)-3-hydroxy-5-methoxypicolinamide;
(R)—N-(5-cyano-2,3-dihydro-1H-inden-1-yl)-5-fluoro-3-hydroxypicolinamide;
(R)-5-chloro-N-(5-cyano-2,3-dihydro-1H-inden-1-yl)-3-hydroxypicolinamide;
(R)—N-(5-cyano-2,3-dihydro-1H-inden-1-yl)-3-hydroxy-5-(trifluoromethyl)picolinamide;
(R)—N-(5-cyano-2,3-dihydro-1H-inden-1-yl)-3-hydroxy-5-methoxypicolinamide;
(R)-5-cyano-N-(5-cyano-2,3-dihydro-1H-inden-1-yl)-3-hydroxypicolinamide;
N-(4-cyano-2-methylbenzyl)-5-fluoro-3-hydroxypicolinamide;
5-chloro-N-(4-cyano-2-methylbenzyl)-3-hydroxypicolinamide;
N-(4-cyano-2-methylbenzyl)-3-hydroxy-5-(trifluoromethyl)picolinamide;
N-(4-cyano-2-methylbenzyl)-3-hydroxy-5-methoxypicolinamide;
N-(1-(4-cyanophenyl)ethyl)-3-hydroxypicolinamide;
N-(4-cyano-2,6-dimethylbenzyl)-3-hydroxy-5-((3-methoxypropyl)amino)picolinamide;
N-(4-cyano-2,6-dimethylbenzyl)-5-(4-ethylpiperazin-1-yl)-3-hydroxypicolinamide;
N-(4-cyano-2,6-dimethylbenzyl)-5-(4-(2,2-difluoroethyl)piperazin-1-yl)-3-hydroxypicolinamide; and
a pharmaceutically acceptable salt of any one of the aforementioned compounds.

24. A pharmaceutical composition comprising:
a compound or pharmaceutically acceptable salt as defined in claim 1; and
a pharmaceutically acceptable excipient.

25-30. (canceled)

31. A method of treating a disease, disorder or condition in a subject, the method comprising administering to the subject a compound or pharmaceutically acceptable salt as defined in claim 1, wherein the disease, disorder or condition is selected from stroke, myocardial infarction, congestive heart failure, atherosclerosis, chronic venous insufficiency, cardiac cirrhosis, acute decompensated heart failure, heart failure following a heart attack, peripheral artery disease, occlusive artery disease, diabetes, hyperglycemia, insulin resistance, metabolic syndrome X, impaired glucose tolerance, non-alcoholic liver steatosis, anemia, chronic obstructive pulmonary disease, pulmonary embolism, pulmonary hypertension, mountain sickness, acute respiratory failure, interstitial lung disease, idiopathic pulmonary fibrosis, desquamative interstitial pneumonia, nonspecific interstitial pneumonia, cryptogenic organizing pneumonia, respiratory bronchiolitis-associated interstitial lung disease, acute interstitial pneumonia, lymphoid interstitial pneumonia, acute kidney failure, acute kidney injury, chronic kidney disease, renal ischemia reperfusion injury, hepatic ischemia reperfusion injury, diabetic foot ulcers, pressure ulcers, venous ulcers, arterial ulcers, epidermolysis bullosa, pemphigus, and Sjogren's Syndrome, and cancer.

32. A combination comprising a compound or pharmaceutically acceptable salt as defined in claim 1, and at least one additional pharmacologically active agent.

33. A pharmaceutical composition comprising:
N-(4-cyanobenzyl)-3-hydroxypicolinamide or a pharmaceutically acceptable salt thereof; and
a pharmaceutically acceptable excipient.

34-41. (canceled)
